(19) United States
(12) Patent Application Publication
(10) Pub. No.: US 2024/0315681 A1
(43) Pub. Date: Sep. 26, 2024

(54) SUPPORTING APPARATUS, AND MANUFACTURING METHOD FOR SUPPORTING APPARATUS

(71) Applicant: CANON KABUSHIKI KAISHA, Tokyo (JP)

(72) Inventors: KATSUHIKO OBA, Kanagawa (JP); YUSUKE NIIKAWA, Kanagawa (JP); ISAO MATSUOKA, Kanagawa (JP)

(21) Appl. No.: 18/735,851

(22) Filed: Jun. 6, 2024

Related U.S. Application Data
(63) Continuation of application No. PCT/JP2022/043288, filed on Nov. 24, 2022.

(30) Foreign Application Priority Data
Dec. 10, 2021 (JP) 2021-200579

(51) Int. Cl. A61B 17/00

(52) U.S. Cl. CPC A61B 17/00234; A61B 2017/00398

(57) ABSTRACT

A supporting apparatus configured to support a unit including a bend body includes (i) a drive member configured to rotate around a rotational axis, (ii) a transmission member configured to be moved in a direction of the rotational axis by rotation of the drive member, and (iii) a driven member for bending the bend body, the driven member being configured to be moved in the direction of the rotational axis by movement of the transmission member. The drive member and the driven member are supported by a first supporting member and a second supporting member having the same shape as the first supporting member. The first supporting member and the second supporting member are engaged with a coupling member.

### CROSS-REFERENCE TO RELATED APPLICATIONS

This application is a Continuation of International Patent Application No. PCT/JP2022/043288, filed Nov. 24, 2022, which claims the benefit of Japanese Patent Application No. 2021-200579, filed Dec. 10, 2021, both of which are hereby incorporated by reference herein in their entirety.

### TECHNICAL FIELD

The present invention relates to a supporting apparatus for a unit having a bend body and a method of manufacturing a supporting apparatus.

### BACKGROUND ART

There is known a configuration that supports a unit having a bend body and that causes the unit to linearly move in a predetermined direction. Japanese Patent No. 4637355 describes a catheter system including a drive assembly to which a catheter assembly is detachably attached. Japanese Patent No. 4637355 describes a configuration in which a drive chassis linearly moves upon receiving rotation of a drive screw.

### CITATION LIST

### Patent Literature

PTL 1 Japanese Patent No. 4637355

In a configuration in which a rotating drive member moves a driven member for bending a bend body in a direction of a rotational axis of the drive member, the drive member and the driven member are preferably parallel to each other in order to accurately move the driven member.

### SUMMARY OF INVENTION

An object of the present invention is to provide a supporting apparatus configured to support a unit including a bend body and capable of accurately positioning a drive member and a driven member. Another object of the present invention is to provide a manufacturing method of manufacturing the supporting apparatus.

One of the inventions according to the subject application is a supporting apparatus configured to support a unit including a bend body. The supporting apparatus includes a drive member configured to rotate around a rotational axis, a transmission member coupled to the drive member and configured to be moved in a direction of the rotational axis by rotation of the drive member, a driven member for bending the bend body, the driven member being coupled to the transmission member and configured to be moved in the direction of the rotational axis by movement of the transmission member, a coupling member, a first supporting member having a first supporting portion that supports the drive member, a second supporting portion that supports the driven member, and a first engaging portion that is engaged with the coupling member, and a second supporting member having the same shape as the first supporting member, the second supporting member having (i) a third supporting portion that corresponds to the first supporting portion and that supports the drive member, (ii) a fourth supporting portion that corresponds to the second supporting portion and that supports the driven member, and (iii) a second engaging portion that corresponds to the first engaging portion and that is engaged with the coupling member, the second supporting member being disposed at a location away from the first supporting member in the direction of the rotational axis.

One of the inventions according to the subject application is a manufacturing method for a supporting apparatus configured to support a unit including a bend body, the supporting apparatus including a drive member configured to rotate around a rotational axis, a transmission member coupled to the drive member and configured to be moved in a direction of the rotational axis by rotation of the drive member, a driven member for bending the bend body, the driven member being coupled to the transmission member and configured to be moved in the direction of the rotational axis by movement of the transmission member, and a coupling member. The manufacturing method includes a step of manufacturing a first supporting member having a first supporting portion, a second supporting portion, and a first engaging portion, a step of manufacturing a second supporting member having (i) a third supporting portion that corresponds to the first supporting portion, (ii) a fourth supporting portion that corresponds to the second supporting portion, and (iii) a second engaging portion that corresponds to the first engaging portion, the second supporting member having the same shape as the first supporting member, and a step of assembling the drive member, the driven member, the first supporting member, the second supporting member, and the coupling member such that the coupling member is engaged with the first engaging portion and the second engaging portion, the drive member is supported by the first supporting portion and the third supporting portion, and the driven member is supported by the second supporting portion and the fourth supporting portion.

Further features of the present invention will become apparent from the following description of exemplary embodiments with reference to the attached drawings.

### BRIEF DESCRIPTION OF DRAWINGS

FIG. 1 is an overall view of a medical system.

FIG. 2 is a perspective view that shows a medical device and a support.

FIG. 3A is a view that illustrates a catheter.

FIG. 3B is a view that illustrates the catheter.

FIG. 4A is a view that illustrates a catheter unit.

FIG. 4B is a view that illustrates the catheter unit.

FIG. 5A is a view that illustrates a base unit and a wire drive portion.

FIG. 5B is a view that illustrates the base unit and the wire drive portion.

FIG. 5C is a view that illustrates the base unit and the wire drive portion.

FIG. 6A is a view that illustrates the wire drive portion, a coupling device, and a bend drive portion.

FIG. 6B is a view that illustrates the wire drive portion, the coupling device, and the bend drive portion.

FIG. 6C is a view that illustrates the wire drive portion, the coupling device, and the bend drive portion.

FIG. 7A is a view that illustrates a support configuration for a motor shaft and a tractor support shaft.

FIG. 7B is a view that illustrates the support configuration for the motor shaft and the tractor support shaft.

FIG. 7C is a view that illustrates the support configuration for the motor shaft and the tractor support shaft.

FIG. 8A is a view that illustrates a first bearing frame and a second bearing frame.

FIG. 8B is a view that illustrates the first bearing frame and the second bearing frame.

FIG. 9A is a view that illustrates a step of manufacturing the first bearing frame and the second bearing frame.

FIG. 9B is a view that illustrates the step of manufacturing the first bearing frame and the second bearing frame.

FIG. 9C is a view that illustrates the step of manufacturing the first bearing frame and the second bearing frame.

FIG. 9D is a view that illustrates the step of manufacturing the first bearing frame and the second bearing frame.

FIG. 10A is a view that illustrates attachment of the catheter unit.

FIG. 10B is a view that illustrates attachment of the catheter unit.

FIG. 11A is a view that illustrates coupling of the catheter unit with the base unit.

FIG. 11B is a view that illustrates coupling of the catheter unit with the base unit.

FIG. 12 is an exploded view that illustrates coupling of the catheter unit with the base unit.

FIG. 13 is a view that illustrates fixing of a drive wire by the coupling portion.

FIG. 14 is a view that illustrates fixing of the drive wire by the coupling portion.

FIG. 15 is a view that illustrates fixing of the drive wire by the coupling portion.

FIG. 16 is a view that illustrates fixing of the drive wire by the coupling portion.

FIG. 17 is a view that illustrates fixing of the drive wire by the coupling portion.

FIG. 18A is a view that illustrates the catheter unit and the base unit.

FIG. 18B is a view that illustrates the catheter unit and the base unit.

FIG. 18C is a view that illustrates the catheter unit and the base unit.

FIG. 19A is a view that illustrates operations of an operating portion.

FIG. 19B is a view that illustrates operations of the operating portion.

FIG. 19C is a view that illustrates operations of the operating portion.

FIG. 20A is a sectional view that illustrates operations of the operating portion.

FIG. 20B is a sectional view that illustrates operations of the operating portion.

FIG. 20C is a sectional view that illustrates operations of the operating portion.

### DESCRIPTION OF EMBODIMENTS

Hereinafter, the configuration of the present invention will be illustrated with reference to the drawings. The dimensions, materials, and shapes of components that will be described in the present embodiments, the arrangement of the components, and the like, should be changed as needed depending on the configuration of an apparatus to which the present invention is applied, various conditions, or the like.

### First Embodiment

### Medical System and Medical Device

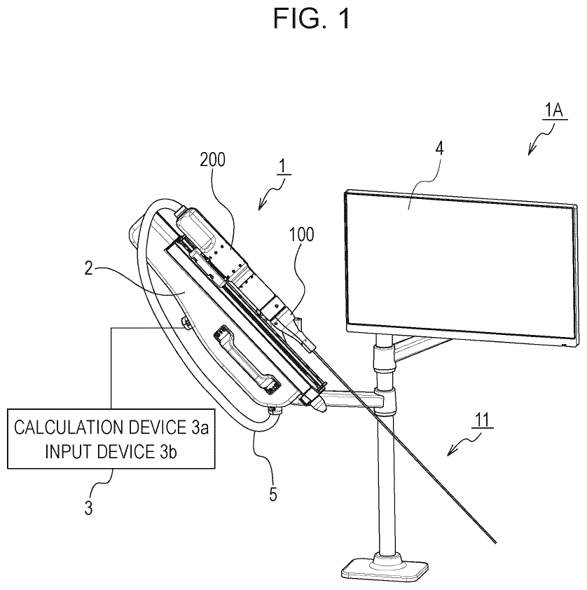

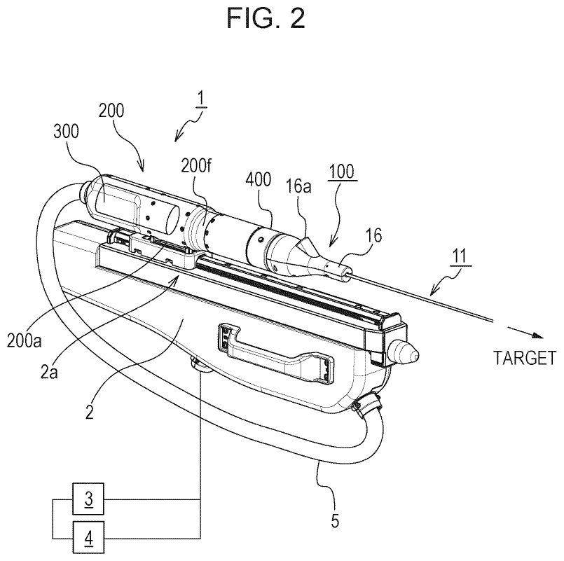

A medical system 1A and a medical device 1 will be described with reference to FIGS. 1 and 2. FIG. 1 is an overall view of the medical system 1A. FIG. 2 is a perspective view that shows the medical device 1 and a support 2.

The medical system 1A includes the medical device 1, the support 2 to which the medical device 1 is attached, and a controller 3 that controls the medical device 1. In the present embodiment, the medical system 1A includes a monitor 4 serving as a display apparatus.

The medical device 1 includes a catheter unit (bendable unit) 100 including a catheter 11 serving as a bendable body, and a base unit (a drive unit or an attached unit) 200. The catheter unit 100 is configured to be detachably attachable to the base unit 200.

In the present embodiment, a user of the medical system 1A and the medical device 1 is able to do some work, such as observing the inside of a target, collecting various samples from the inside of the target, and treating the inside of the target, by inserting the catheter 11 into the inside of the target. As one of embodiments, a user is able to insert the catheter 11 into the inside of a patient as a target. Specifically, by inserting the catheter 11 into a bronchus via the oral cavity or nasal cavity of a patient, it is possible to do some work, such as observation, collection, and removal of a lung tissue.

The catheter 11 can be used as a guide (sheath) that guides a medical tool for doing the above work. Examples of the medical tool (tool) include an endoscope, a forceps, and an ablation device. The catheter 11 itself may have the functions of the above-described medical tools.

In the present embodiment, the controller 3 includes a calculation device 3a and an input device 3b. The input device 3b receives a command or input for operating the catheter 11. The calculation device 3a includes a storage that stores a program and various data for controlling the catheter 11, a random access memory, and a central processing unit for running the program. The controller 3 may include an output section that outputs a signal for displaying an image on the monitor 4.

As shown in FIG. 2, in the present embodiment, the medical device 1 is electrically connected to the controller 3 via the support 2 and a cable 5 that couples the base unit 200 of the medical device 1 with the support 2. The medical device 1 and the controller 3 may be directly connected by a cable. The medical device 1 and the controller 3 may be wirelessly connected to each other.

The medical device 1 is detachably attached to the support 2 via the base unit 200. More specifically, in the medical device 1, an attachment portion (connecting portion) 200a of the base unit 200 is detachably attached to a movable stage (receiving portion) 2a of the support 2. Even in a state where the attachment portion 200a of the medical device 1 is detached from the movable stage 2a, connection of the medical device 1 with the controller 3 is maintained such that the medical device 1 is controllable by the controller 3. In the present embodiment, even in a state where the attachment portion 200a of the medical device 1 is detached from the movable stage 2a, the medical device 1 and the support 2 are connected by the cable 5.

A user is able to manually move the medical device 1 in a state where the medical device 1 is detached from the support 2 (a state where the medical device 1 is detached from the movable stage 2a) and insert the catheter 11 into the inside of a target.

A user is able to use the medical device 1 in a state where the catheter 11 is inserted in a target and the medical device 1 is attached to the support 2. Specifically, when the movable stage 2a moves in a state where the medical device 1 is attached to the movable stage 2a, the medical device 1 moves. Then, an operation to move the catheter 11 in a direction to be inserted into the target and an operation to move the catheter 11 in a direction to be pulled out from the target are performed. Movement of the movable stage 2a is controlled by the controller 3.

The attachment portion 200a of the base unit 200 includes an unlock switch (not shown) and a detachment switch (not shown). In a state where the attachment portion 200a is attached to the movable stage 2a, a user is able to manually move the medical device 1 along a guide direction of the movable stage 2a while holding down the unlock switch. In other words, the movable stage 2a includes a guide configuration to guide movement of the medical device 1. When the user stops pressing the unlock switch, the medical device 1 is fixed to the movable stage 2a. On the other hand, when the detachment switch is pressed in a state where the attachment portion 200a is attached to the movable stage 2a, a user is able to detach the medical device 1 from the movable stage 2a.

A single switch may have the function of the unlock switch and the function of the detachment switch. When the unlock switch is provided with a mechanism of switching between a pressed down state and a non-pressed down state, a user does not need to hold down the unlock switch when manually sliding the medical device 1.

In a state where the attachment portion 200a is attached to the movable stage 2a and the unlock switch or the detachment switch is not pressed, the medical device 1 is fixed to the movable stage 2a and is moved by the movable stage 2a driven by a motor (not shown) .

The medical device 1 includes a wire drive portion (a linear member drive portion, a line drive portion, or a main body drive portion) 300 for actuating the catheter 11. In the present embodiment, the medical device 1 is a robot catheter device that actuates the catheter 11 with the wire drive portion 300 controlled by the controller 3.

The controller 3 can control the wire drive portion 300 and perform an operation to bend the catheter 11. In the present embodiment, the wire drive portion 300 is incorporated in the base unit 200. More specifically, the base unit 200 includes a base housing 200f that accommodates the wire drive portion 300. In other words, the base unit 200 includes the wire drive portion 300. The wire drive portion 300 and the base unit 200 may be collectively referred to as a catheter drive apparatus (a base apparatus or a main body).

In an extending direction of the catheter 11, an end portion at which the tip end of the catheter 11 to be inserted into a target is disposed is referred to as a distal end. In the extending direction of the catheter 11, an opposite side to the distal end is referred to as a proximal end.

The catheter unit 100 has a proximal end cover 16 that covers the proximal end side of the catheter 11. The proximal end cover 16 has a tool hole 16a. A medical tool is allowed to be inserted into the catheter 11 via the tool hole 16a.

As described above, in the present embodiment, the catheter 11 has the function of a guide device for guiding a medical tool to a desired location inside a target.

For example, in a state where an endoscope is inserted in the catheter 11, the catheter 11 is inserted to an intended location inside a target. At this time, at least one of manual operation of a user, movement of the movable stage 2a, and actuation of the catheter 11 with the wire drive portion 300 is used. After the catheter 11 reaches the intended location, the endoscope is pulled out from the catheter 11 via the tool hole 16a. Then, a medical tool is inserted through the tool hole 16a, and some work, such as collecting various samples from the inside of the target and treating the inside of the target, is performed.

As will be described later, the catheter unit 100 is detachably attached to the catheter drive apparatus (a base apparatus or a main body), more specifically, the base unit 200. After the medical device 1 is used, a user is able to detach the catheter unit 100 from the base unit 200, attach a new catheter unit 100 to the base unit 200, and use the medical device 1 again.

As shown in FIG. 2, the medical device 1 includes an operating portion 400. In the present embodiment, the operating portion 400 is provided in the catheter unit 100. The operating portion 400 is operated by a user when the catheter unit 100 is fixed to the base unit 200 or when the catheter unit 100 is detached from the base unit 200.

By connecting the endoscope inserted in the catheter 11 with the monitor 4, the monitor 4 can display an image taken by the endoscope on the monitor 4. By connecting the monitor 4 with the controller 3, the status of the medical device 1 and information related to control over the medical device 1 can be displayed on the monitor 4. For example, the location of the catheter 11 inside a target or information related to navigation for the catheter 11 inside a target can be displayed on the monitor 4. The monitor 4 and both the controller 3 and the endoscope may be connected by wire or may be connected by wireless. The monitor 4 and the controller 3 may be directly connected via the support 2.

### Catheter

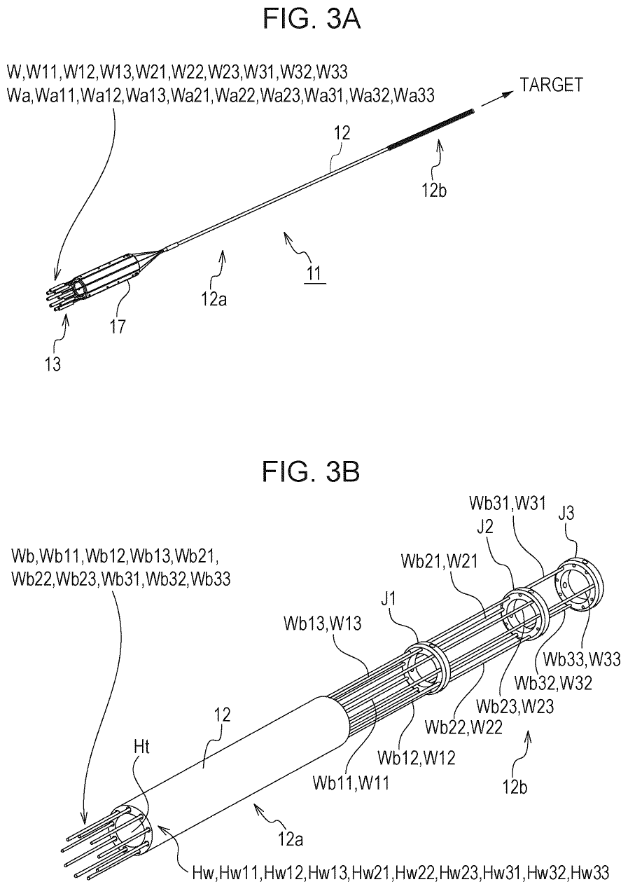

The catheter 11 serving as a bendable body will be described with reference to FIGS. 3A and 3B. FIGS. 3A and 3B are views that illustrate the catheter 11. FIG. 3A is a view that illustrates the whole of the catheter 11. FIG. 3B is an enlarged view of the catheter 11.

The catheter 11 includes a bend portion (a bend body or a catheter main body) 12 and a bend drive portion (catheter drive portion) 13 configured to bend the bend portion 12. The bend drive portion 13 is configured to bend the bend portion 12 upon receiving the driving force of the wire drive portion 300 via a coupling device 21 (described later).

The catheter 11 extends along the direction in which the catheter 11 is inserted into a target. The extending direction (longitudinal direction) of the catheter 11 is the same as an extending direction (longitudinal direction) of the bend portion 12 and an extending direction (longitudinal direction) of each of first to ninth drive wires (W11 to W33) (described later).

The bend drive portion 13 includes the plurality of drive wires (drive lines, linear members, or linear actuators) connected to the bend portion 12. Specifically, the bend drive portion 13 includes the first drive wire W11, the second drive wire W12, the third drive wire W13, the fourth drive wire W21, the fifth drive wire W22, the sixth drive wire W23, the seventh drive wire W31, the eighth drive wire W32, and the ninth drive wire W33.

Each of the first to ninth drive wires (W11 to W33) includes a held portion (a held shaft or a rod) Wa. Specifically, the first drive wire W11 includes the first held portion Wa11. The second drive wire W12 includes the second held portion Wa12. The third drive wire W13 includes the third held portion Wa13. The fourth drive wire W21 includes the fourth held portion Wa21. The fifth drive wire W22 includes the fifth held portion Wa22. The sixth drive wire W23 includes the sixth held portion Wa23. The seventh drive wire W31 includes the seventh held portion Wa31. The eighth drive wire W32 includes the eighth held portion Wa32. The ninth drive wire W33 includes the ninth held portion Wa33.

In the present embodiment, each of the first to ninth held portions (Wa11 to Wa33) has the same shape.

Each of the first to ninth drive wires (W11 to W33) includes a flexible wire body (a line body or a linear body) Wb. Specifically, the first drive wire W11 includes the first wire body Wb11. The second drive wire W12 includes the second wire body Wb12. The third drive wire W13 includes the third wire body Wb13. The fourth drive wire W21 includes the fourth wire body Wb21. The fifth drive wire W22 includes the fifth wire body Wb22. The sixth drive wire W23 includes the sixth wire body Wb23. The seventh drive wire W31 includes the seventh wire body Wb31. The eighth drive wire W32 includes the eighth wire body Wb32. The ninth drive wire W33 includes the ninth wire body Wb33.

In the present embodiment, each of the first to third wire bodies (Wb11 to Wb13) has the same shape. Each of the fourth to sixth wire bodies (Wb21 to Wb23) has the same shape. Each of the seventh to ninth wire bodies (Wb31 to Wb33) has the same shape. In the present embodiment, the first to ninth wire bodies (Wb11 to Wb33) have the same shape.

Each of the first to ninth held portions (Wa11 to Wa33) is fixed to a corresponding one of the first to ninth wire bodies (Wb11 to Wb33) at the proximal end of the corresponding one of the first to ninth wire bodies (Wb11 to Wb33).

The first to ninth drive wires (W11 to W33) are inserted into the bend portion 12 via a wire guide 17 and fixed.

In the present embodiment, the material of each of the first to ninth drive wires (W11 to W33) is a metal. The material of each of the first to ninth drive wires (W11 to W33) may be a resin. The material of each of the first to ninth drive wires (W11 to W33) may include metal and resin.

Of the first to ninth drive wires (W11 to W33), a selected one may be referred to as a drive wire W. In the present embodiment, each of the first to ninth drive wires (W11 to W33) has the same shape except for the length of each of the first to ninth wire bodies (Wb11 to Wb33).

In the present embodiment, the bend portion 12 has flexibility and is a tubular member having a passage Ht for inserting a medical tool.

A wall of the bend portion 12 includes a plurality of wire holes for respectively passing the first to ninth drive wires (W11 to W33). Specifically, the wall of the bend portion 12 includes the first wire hole Hw11, the second wire hole Hw12, the third wire hole Hw13, the fourth wire hole Hw21, the fifth wire hole Hw22, the sixth wire hole Hw23, the seventh wire hole Hw31, the eighth wire hole Hw32, and the ninth wire hole Hw33. The first to ninth wire holes Hw (Hw11 to Hw33) are respectively in correspondence with the first to ninth drive wires (W11 to W33). The numeral suffixed to the sign Hw represents the numeral of a corresponding one of the drive wires. For example, the first drive wire W11 is inserted into the first wire hole Hw11.

Of the first to ninth wire holes (Hw11 to Hw33), a selected one may be referred to as a wire hole Hw. In the present embodiment, each of the first to ninth wire holes (Hw11 to Hw33) has the same shape.

The bend portion 12 has an intermediate region 12a and a bend region 12b. The bend region 12b is disposed at the distal end of the bend portion 12. A first guide ring J1, a second guide ring J2, and a third guide ring J3 are disposed in the bend region 12b. The bend region 12b means a region capable of controlling the degree and direction of bending of the bend portion 12 by moving the first guide ring J1, the second guide ring J2, and the third guide ring J3 with the bend drive portion 13. FIG. 3B is a view drawn by omitting part of the bend portion 12 covering the first to third guide rings (J1 to J3).

In the present embodiment, the bend portion 12 includes a plurality of sub-rings (not shown). In the bend region 12b, the first guide ring J1, the second guide J2, and the third guide ring J3 are fixed to the wall of the bend portion 12. In the present embodiment, the sub-rings are disposed between the first guide ring J1 and the second guide ring J2, and between the second guide ring J2 and the third guide ring J3.

A medical tool is guided to the tip end of the catheter 11 by the passage Ht, the first to third guide rings (J1 to J3), and the plurality of sub-rings.

Each of the first to ninth drive wires (W11 to W33) is fixed to a corresponding one of the first to third guide rings (J1 to J3) through the intermediate region 12a.

Specifically, the first drive wire W11, the second drive wire W12, and the third drive wire W13 are fixed to the first guide ring J1. The fourth drive wire W21, the fifth drive wire W22, and the sixth drive wire W23 extend through the first guide ring J1 and the plurality of sub-rings and are fixed to the second guide ring J2. The seventh drive wire W31, the eighth drive wire W32, and the ninth drive wire W33 extend through the first guide ring J1, the second guide ring J2, and the plurality of sub-rings and are fixed to the third guide ring J3.

The medical device 1 is capable of bending the bend portion 12 in a direction that intersects with the extending direction of the catheter 11 by actuating the bend drive portion 13 with the wire drive portion 300. Specifically, by moving each of the first to ninth drive wires (W11 to W33) in the extending direction of the bend portion 12, the bend region 12b of the bend portion 12 is bent in a direction that intersects with the extending direction via the first to third guide rings (J1 to J3).

A user is able to insert the catheter 11 to an intended part inside a target by using at least any one of moving the medical device 1 manually or with the movable stage 2a and bending the bend portion 12.

In the present embodiment, the bend portion 12 is bent by moving the first to third guide rings (J1 to J3) with the first to ninth drive wires (W11 to W33); however, the present invention is not limited to this configuration. Any one or two of the first to third guide rings (J1 to J3) and the drive wires fixed to them may be omitted.

For example, the catheter 11 may have such a configuration that the seventh to ninth drive wires (W31 to W33) and the third guide ring J3 are provided and the first to sixth drive wires (W11 to W23) and the first and second guide rings (J1, J2) are omitted. Alternatively, the catheter 11 may have such a configuration that the fourth to ninth drive wires (W21 to W33) and the second and third guide rings (J2, J3) are provided and the first to third drive wires (W11 to W13) and the first guide ring J1 are omitted.

Alternatively, the catheter 11 may have such a configuration that a single guide ring is driven by two drive wires. In this case as well, the number of guide rings may be one or may be more than one.

### Catheter Unit

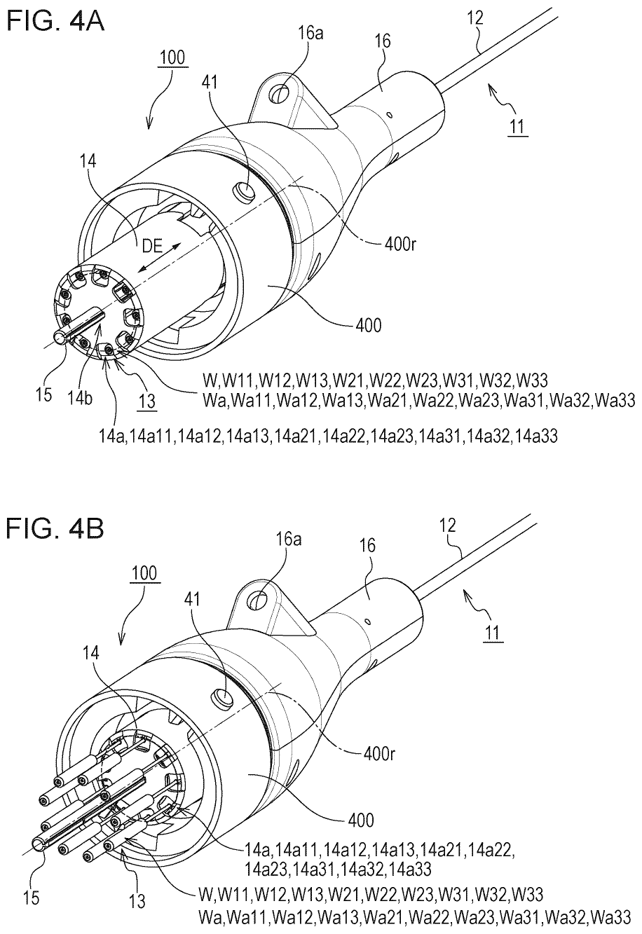

The catheter unit 100 will be described with reference to FIGS. 4A and 4B.

FIGS. 4A and 4B are views that illustrate the catheter unit 100. FIG. 4A is a view that illustrates the catheter unit 100 in a state where a wire cover 14 (described later) is at a cover position. FIG. 4B is a view that illustrates the catheter unit 100 in a state where the wire cover 14 (described later) is at an exposed position.

The catheter unit 100 includes the catheter 11 including the bend portion 12 and the bend drive portion 13, and a proximal end cover 16 that supports the proximal end of the catheter 11. The catheter unit 100 includes the cover (wire cover) 14 for covering and protecting the first to ninth drive wires (W11 to W33) serving as the plurality of drive wires.

The catheter unit 100 is detachably attachable to the base unit 200 along an attaching and detaching direction DE. The direction in which the catheter unit 100 is attached to the base unit 200 and the direction in which the catheter unit 100 is detached from the base unit 200 are parallel to the attaching and detaching direction DE.

The proximal end cover (a frame, a bend portion housing, or a catheter housing) 16 is a cover covering part of the catheter 11. The proximal end cover 16 has the tool hole 16a for inserting a medical tool into the passage Ht of the bend portion 12.

The wire cover 14 has a plurality of exposure holes (wire cover holes or cover holes) for respectively passing the first to ninth drive wires (W11 to W33). The wire cover 14 has the first exposure hole 14a11, the second exposure hole 14a12, the third exposure hole 14a13, the fourth exposure hole 14a21, the fifth exposure hole 14a22, the sixth exposure hole 14a23, the seventh exposure hole 14a31, the eighth exposure hole 14a32, and the ninth exposure hole 14a33. The first to ninth exposure holes (14a11 to 14a33) are respectively in correspondence with the first to ninth drive wires (W11 to W33). The numeral suffixed to the sign 14a represents the numeral of a corresponding one of the drive wires. For example, the first drive wire W11 is inserted into the first exposure hole 14a11.

Of the first to ninth exposure holes (14a11 to 14a33), a selected one may be referred to as an exposure hole 14a. In the present embodiment, each of the first to ninth exposure holes (14a11 to 14a33) has the same shape.

The wire cover 14 can be moved to a cover position (see FIG. 4A) where the first to ninth drive wires (W11 to W33) are covered and a cover retracted position (see FIG. 4B) retracted from the cover position. The cover retracted position may also be referred to as an exposed position where the first to ninth drive wires (W11 to W33) are exposed.

Before the catheter unit 100 is attached to the base unit 200, the wire cover 14 is located at the cover position. When the catheter unit 100 is attached to the base unit 200, the wire cover 14 moves from the cover position to the exposed position along the attaching and detaching direction DE.

In the present embodiment, after the wire cover 14 is moved from the cover position to the exposed position, the wire cover 14 is retained at the exposed position. Therefore, even when the catheter unit 100 is attached to the base unit 200 and then the catheter unit 100 is detached from the base unit 200, the wire cover 14 is retained at the exposed position.

However, after the wire cover 14 is moved from the cover position to the exposed position, the wire cover 14 may be configured to return to the cover position. For example, the catheter unit 100 may include an urging member that urges the wire cover 14 from the exposed position toward the cover position. In this case, when the catheter unit 100 is detached from the base unit 200 after the catheter unit 100 is attached to the base unit 200, the wire cover 14 is moved from the exposed position to the cover position.

When the wire cover 14 is at the exposed position, the first to ninth held portions (Wa11 to Wa33) of the first to ninth drive wires (W11 to W33) are exposed. As a result, coupling of the bend drive portion 13 with the coupling device 21 (described later) is allowed. When the wire cover 14 is at the exposed position, the first to ninth held portions (Wa11 to Wa33) of the first to ninth drive wires (W11 to W33) protrude from the first to ninth exposure holes (14a11 to 14a33). More specifically, the first to ninth held portions (Wa11 to Wa33) protrude from the first to ninth exposure holes (14a11 to 14a33) in an attachment direction Da (described later).

As shown in FIG. 4B, the first to ninth drive wires (W11 to W33) are arranged along a circle (imaginary circle) having a predetermined radius.

In the present embodiment, the catheter unit 100 includes a key shaft (a key or a catheter-side key) 15. In the present embodiment, the key shaft 15 extends in the attaching and detaching direction DE. The wire cover 14 has a shaft hole 14b through which the key shaft 15 extends. The key shaft 15 is engageable with a key receiving portion 22 (described later). When the key shaft 15 is engaged with the key receiving portion 22, movement of the catheter unit 100 with respect to the base unit 200 is limited within a predetermined range in the circumferential direction of the circle (imaginary circle) along which the first to ninth drive wires (W11 to W33) are arranged.

In the present embodiment, when viewed in the attaching and detaching direction DE, the first to ninth drive wires (W11 to W33) are disposed outside the key shaft 15 so as to surround the key shaft 15. In other words, the key shaft 15 is disposed inside the circle (imaginary circle) along which the first to ninth drive wires (W11 to W33) are arranged. Therefore, the key shaft 15 and the first to ninth drive wires (W11 to W33) can be disposed in a space-saving manner.

In the present embodiment, the catheter unit 100 includes the operating portion 400. The operating portion 400 is configured to be movable (rotatable) with respect to the proximal end cover 16 and the bend drive portion 13. The operating portion 400 is rotatable 400 around a rotation axis 400r. The rotation axis 400r of the operating portion 400 extends in the attaching and detaching direction DE.

In a state where the catheter unit 100 is attached to the base unit 200, the operating portion 400 is configured to be movable (rotatable) with respect to the base unit 200. More specifically, the operating portion 400 is configured to be movable (rotatable) with respect to the base housing 200f, the wire drive portion 300, and the coupling device 21 (described later).

### Base Unit

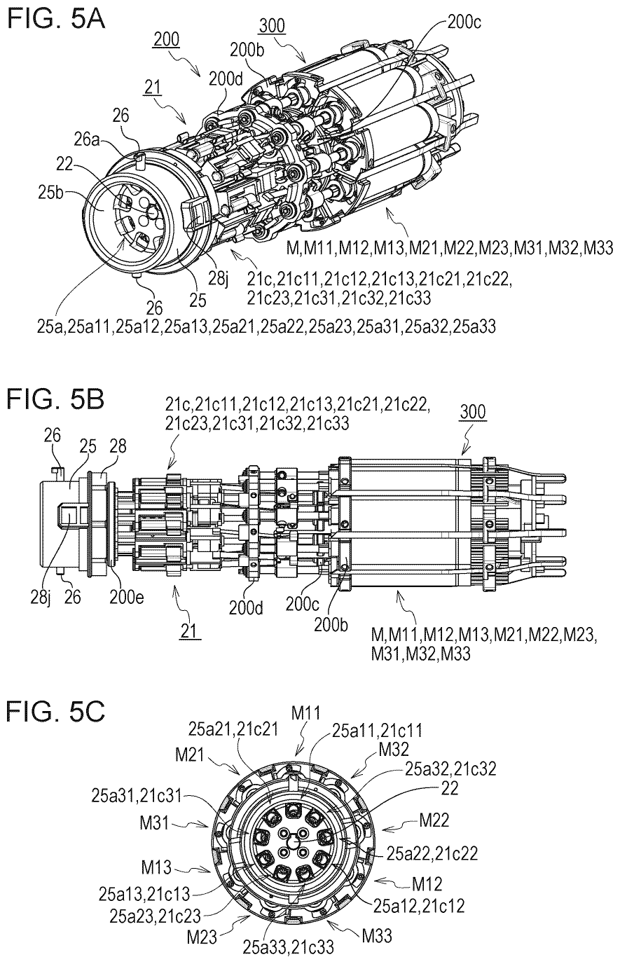

The base unit 200 and the wire drive portion 300 will be described with reference to FIGS. 5A to 5C.

FIGS. 5A to 5C are views that illustrate the base unit 200 and the wire drive portion 300. FIG. 5A is a perspective view that shows the internal structure of the base unit 200. FIG. 5B is a side view that shows the internal structure of the base unit 200. FIG. 5C is a view of the base unit 200 when viewed along the attaching and detaching direction DE.

As described above, the medical device 1 includes the base unit 200 and the wire drive portion 300. In the present embodiment, the wire drive portion 300 is accommodated in the base housing 200f and is provided inside the base unit 200. In other words, the base unit 200 includes the wire drive portion 300. The base unit 200 including the wire drive portion 300 has the function of a supporting apparatus (a supporting unit or a receiving unit) to which the catheter unit 100 is attachable.

The wire drive portion 300 includes a plurality of driving sources (motors). In the present embodiment, the wire drive portion 300 includes the first driving source M11, the second driving source M12, the third driving source M13, the fourth driving source M21, the fifth driving source M22, the sixth driving source M23, the seventh driving source M31, the eighth driving source M32, and the ninth driving source M33.

Of the first to ninth driving sources (M11 to M33), a selected one may be referred to as a driving source M. In the present embodiment, each of the first to ninth driving sources (M11 to M33) has the same configuration.

The base unit 200 includes the coupling device 21. The coupling device 21 is accommodated in the base housing 200f. The coupling device 21 is connected to the wire drive portion 300. The coupling device 21 includes a plurality of coupling portions. In the present embodiment, the coupling device 21 includes the first coupling portion 21c11, the second coupling portion 21c12, the third coupling portion 21c13, the fourth coupling portion 21c21, the fifth coupling portion 21c22, the sixth coupling portion 21c23, the seventh coupling portion 21c31, the eighth coupling portion 21c32, and the ninth coupling portion 21c33.

Of the first to ninth coupling portions (21c11 to 21c33), a selected one may be referred to as a coupling portion 21c. In the present embodiment, each of the first to ninth coupling portions (21c11 to 21c33) has the same configuration.

Each of the plurality of coupling portions is connected to a corresponding one of the plurality of driving sources and is driven by the corresponding one of the plurality of driving sources. Specifically, the first coupling portion 21c11 is connected to the first driving source M11 and is driven by the first driving source M11. The second coupling portion 21c12 is connected to the second driving source M12 and is driven by the second driving source M12. The third coupling portion 21c13 is connected to the third driving source M13 and is driven by the third driving source M13. The fourth coupling portion 21c21 is connected to the fourth driving source M21 and is driven by the fourth driving source M21. The fifth coupling portion 21c22 is connected to the fifth driving source M22 and is driven by the fifth driving source M22. The sixth coupling portion 21c23 is connected to the sixth driving source M23 and is driven by the sixth driving source M23. The seventh coupling portion 21c31 is connected to the seventh driving source M31 and is driven by the seventh driving source M31. The eighth coupling portion 21c32 is connected to the eighth driving source M32 and is driven by the eighth driving source M32. The ninth coupling portion 21c33 is connected to the ninth driving source M33 and is driven by the ninth driving source M33.

As will be described later, the bend drive portion 13 including the first to ninth drive wires (W11 to W33) is coupled to the coupling device 21. The bend drive portion 13 receives the driving force of the wire drive portion 300 via the coupling device 21 to bend the bend portion 12.

The drive wire W is coupled to the coupling portion 21c via the held portion Wa. Each of the plurality of drive wires is coupled to a corresponding one of the plurality of coupling portions.

Specifically, the first held portion Wa11 of the first drive wire W11 is coupled to the first coupling portion 21c11. The second held portion Wa12 of the second drive wire W12 is coupled to the second coupling portion 21c12. The third held portion Wa13 of the third drive wire W13 is coupled to the third coupling portion 21c13. The fourth held portion Wa21 of the fourth drive wire W21 is coupled to the fourth coupling portion 21c21. The fifth held portion Wa22 of the fifth drive wire W22 is coupled to the fifth coupling portion 21c22. The sixth held portion Wa23 of the sixth drive wire W23 is coupled to the sixth coupling portion 21c23. The seventh held portion Wa31 of the seventh drive wire W31 is coupled to the seventh coupling portion 21c31. The eighth held portion Wa32 of the eighth drive wire W32 is coupled to the eighth coupling portion 21c32. The ninth held portion Wa33 of the ninth drive wire W33 is coupled to the ninth coupling portion 21c33.

The base unit 200 includes a base frame 25. The base frame 25 has a plurality of insertion holes for respectively passing the first to ninth drive wires (W11 to W33). The base frame 25 has the first insertion hole 25a11, the second insertion hole 25a12, the third insertion hole 25a13, the fourth insertion hole 25a21, the fifth insertion hole 25a22, the sixth insertion hole 25a23, the seventh insertion hole 25a31, the eighth insertion hole 25a32, and the ninth insertion hole 25a33. The first to ninth insertion holes (25a11 to 25a33) are respectively in correspondence with the first to ninth drive wires (W11 to W33). The numeral suffixed to the sign 25a represents the numeral of a corresponding one of the drive wires. For example, the first drive wire W11 is inserted into the first insertion hole 25a11.

Of the first to ninth insertion holes (25a11 to 25a33), a selected one may be referred to as an insertion hole 25a. In the present embodiment, each of the first to ninth insertion holes (25a11 to 25a33) has the same shape.

The base frame 25 has an attachment opening 25b into which the wire cover 14 is inserted. The first to ninth insertion holes (25a11 to 25a33) are disposed at the bottom portion of the attachment opening 25b.

In addition, the base unit 200 includes a main frame 200b, a first bearing frame 200c, a second bearing frame 200d, and a third bearing frame 200e. The main frame 200b, the first bearing frame 200c, the second bearing frame 200d, and the third bearing frame 200e are coupled.

The base frame 25 has a key receiving portion (a key hole, a base-side key, or a main body-side key) 22 for receiving the key shaft 15. When the key shaft 15 and the key receiving portion 22 are engaged with each other, the catheter unit 100 is prevented from being attached to the base unit 200 in wrong phase.

When the key shaft 15 is engaged with the key receiving portion 22, movement of the catheter unit 100 with respect to the base unit 200 is limited within a predetermined range in the circumferential direction of the circle (imaginary circle) along which the first to ninth drive wires (W11 to W33) are arranged.

As a result, each of the first to ninth drive wires (W11 to W33) is engaged with a corresponding one of the first to ninth insertion holes (25a11 to 25a33) and a corresponding one of the first to ninth coupling portions (21c11 to 21c33). In other words, engagement of the drive wire W with the insertion hole 25a different from a corresponding one of the insertion holes 25a and the coupling portion 21c different from a corresponding one of the coupling portions 21c is prevented.

A user is able to properly couple each of the first to ninth drive wires (W11 to W33) with a corresponding one of the first to ninth coupling portions (21c11 to 21c33) by engaging the key shaft 15 with the key receiving portion 22. Therefore, a user is able to easily attach the catheter unit 100 to the base unit 200.

In the present embodiment, the key shaft 15 has a protruding portion that protrudes in a direction to intersect with the attaching and detaching direction DE, and the key receiving portion 22 has a recess portion into which the protruding portion is inserted. A position in which the protruding portion and the recess portion are engaged with each other in the circumferential direction is a position in which each of the drive wires W is engaged with a corresponding one of the insertion holes 25a and a corresponding one of the coupling portions 21c.

The key shaft 15 may be disposed in any one of the base unit 200 and the catheter unit 100, and the key receiving portion 22 may be disposed in the other. For example, the key shaft 15 may be disposed at the base unit 200 side, and the key receiving portion 22 may be disposed at the catheter unit 100 side.

The base unit 200 has a joint 28 that includes a joint engagement portion 28j. The base frame 25 includes a lock shaft 26 having a lock protrusion 26a. The functions of them will be described later.

### Coupling of Motor With Drive Wire

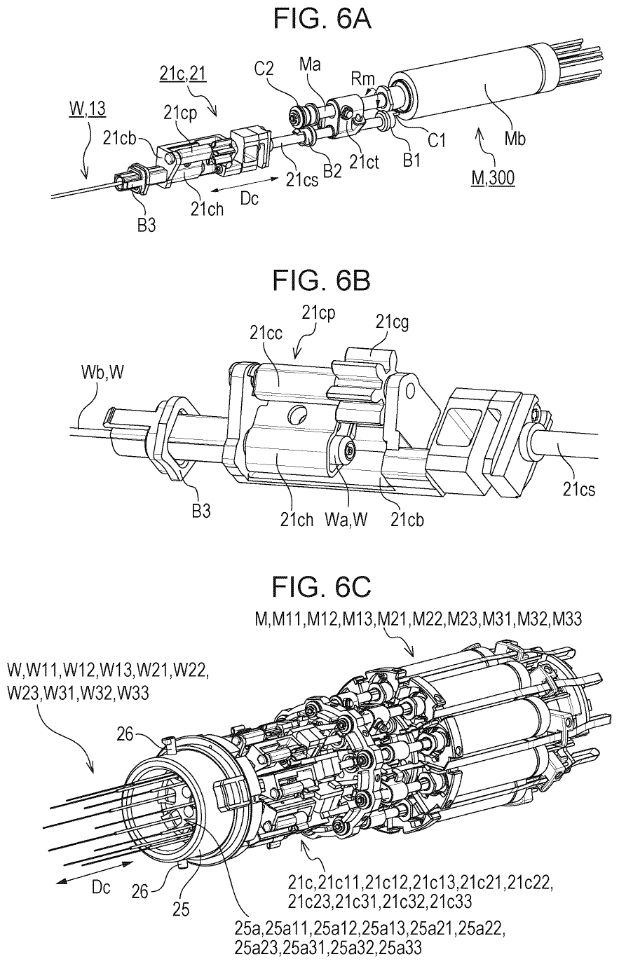

Coupling among the wire drive portion 300, the coupling device 21, and the bend drive portion 13 will be described with reference to FIGS. 6A to 6C.

FIGS. 6A to 6C are views that illustrate the wire drive portion 300, the coupling device 21, and the bend drive portion 13. FIG. 6A is a perspective view of the driving source M, the coupling portion 21c, and the drive wire W. FIG. 6B is an enlarged view of the coupling portion 21c and the drive wire W. FIG. 6C is a perspective view that shows coupling among the wire drive portion 300, the coupling device 21, and the bend drive portion 13.

In the present embodiment, the configuration in which each of the first to ninth drive wires (W11 to W33) is coupled to a corresponding one of the first to ninth coupling portions (21c11 to 21c33) is the same. The configuration in which each of the first to ninth coupling portions (21c11 to 21c33) is connected to a corresponding one of the first to ninth driving sources (M11 to M33) is the same. Therefore, in the following description, the configuration in which the drive wire W, the coupling portion 21c, and the driving source M are connected will be described by using the one drive wire W, the one coupling portion 21c, and the one driving source M.

As shown in FIG. 6A, the driving source M includes a motor shaft (drive member) Ma and a motor main body Mb that rotates the motor shaft Ma in a rotation direction Rm. A spiral groove is provided on the surface of the motor shaft Ma. The motor shaft Ma has a so-called screw shape.

The coupling portion 21c has a tractor (transmission member) 21ct connected to the motor shaft Ma and a tractor support shaft 21cs that supports the tractor 21ct. The tractor support shaft 21cs is connected to a coupling base 21cb.

The coupling portion 21c includes a leaf spring 21ch serving as a holding portion for holding the held portion Wa of the drive wire W. The drive wire W is engaged with the coupling portion 21c through the insertion hole 25a. More specifically, the held portion Wa is engaged with the leaf spring 21ch. As will be described later, the leaf spring 21ch can be placed in a state where the held portion Wa is clamped and fixed (fixed state) and a state where the held portion Wa is released (released state).

The coupling portion 21c includes a pressing member 21cp. The pressing member 21cp has a gear portion 21cg meshed with an internal gear 29 (described later) and a cam 21cc serving as a pressing portion for pressing the leaf spring 21ch.

As will be described later, the cam 21cc can move with respect to the leaf spring 21ch. When the cam 21cc moves, the leaf spring 21ch is switched between the fixed state and the released state.

The coupling portion 21c is supported by a first bearing B1, a second bearing B2, and a third bearing B3. The first bearing B1 is supported by the first bearing frame 200c of the base unit 200 (described later). The second bearing B2 is supported by the second bearing frame 200d of the base unit 200 (described later). The third bearing B3 is supported by the third bearing frame 200e of the base unit 200. Therefore, when the motor shaft Ma rotates in the rotation direction Rm, rotation of the coupling portion 21c around the motor shaft Ma is restricted. The first bearing B1, the second bearing B2, and the third bearing B3 are provided for each of the first to ninth coupling portions (21c11 to 21c33).

The tractor 21ct is coupled to the motor shaft Ma, and the tractor support shaft 21cs is coupled to the tractor 21ct. Since rotation of the coupling portion 21c around the motor shaft Ma is restricted, when the motor shaft Ma rotates, a force along the rotational axis direction of the motor shaft Ma is applied to the tractor 21ct by the spiral groove of the motor shaft Ma.

With rotation of the motor shaft Ma, the tractor 21ct is moved in the direction of the rotational axis of the motor shaft Ma with respect to the motor shaft Ma. On the other hand, the tractor support shaft 21cs is fixed with respect to the tractor 21ct. With movement of the tractor 21ct, the tractor support shaft 21cs moves in the direction of the rotational axis of the motor shaft Ma together with the coupling base 21cb. In other words, the coupling portion 21c moves along the rotational axis direction of the motor shaft Ma (Dc direction). When the coupling portion 21c moves, the drive wire W moves, and the bend portion 12 bends.

In other words, the motor shaft Ma and the tractor 21ct make up a so-called feed screw that converts rotational motion transmitted from the driving source M to linear motion by a screw. In the present embodiment, the motor shaft Ma and the tractor 21ct are slide screws. Alternatively, the motor shaft Ma and the tractor 21ct may be ball screws.

As shown in FIG. 6C, by attaching the catheter unit 100 to the base unit 200, each of the first to ninth drive wires (W11 to W33) and a corresponding one of the first to ninth coupling portions (21c11 to 21c33) are coupled to each other.

The controller 3 is capable of controlling the first to ninth driving sources (M11 to M33) independently of one another. In other words, a selected one driving source of the first to ninth driving sources (M11 to M33) is allowed to independently operate or stop regardless of whether the other driving sources are stopped. In other words, the controller 3 is capable of controlling each of the first to ninth drive wires (W11 to W33) independently of one another. As a result, each of the first to third guide rings (J1 to J3) is controlled independently of one another, and the bend region 12b of the bend portion 12 is allowed to bend in a selected direction.

### Support Configuration of Motor Shaft and Tractor Support Shaft

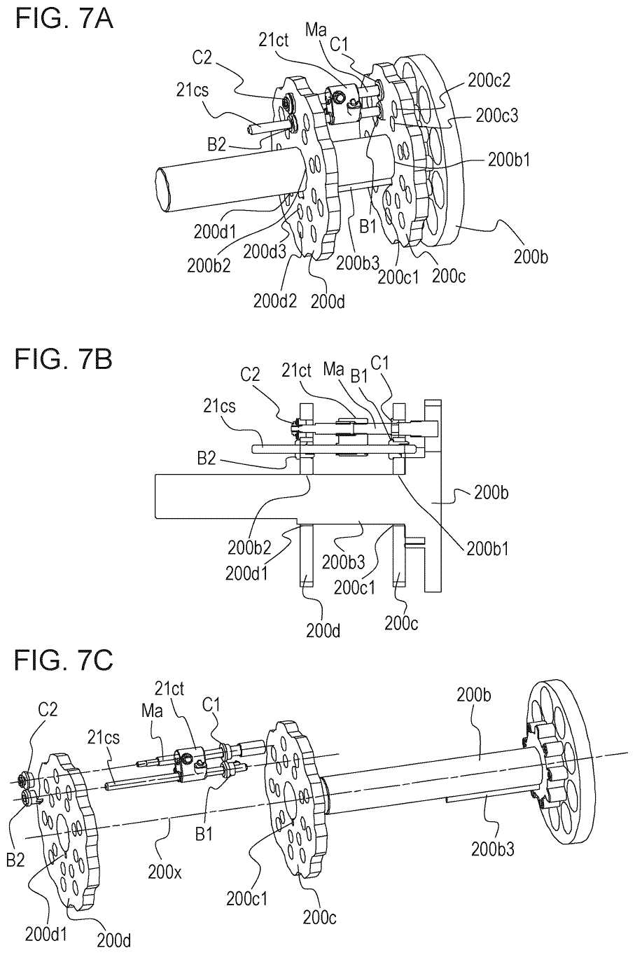

The support configuration for the motor shaft (drive member) Ma and the tractor support shaft (driven member) 21cs will be described with reference to FIGS. 7A to 7C, 8A, 8B, and 9A to 9D.

FIGS. 7A to 7C are views that illustrate a support configuration for the motor shaft Ma and the tractor support shaft 21cs. FIG. 7A is a perspective view of the motor shaft Ma and the tractor support shaft 21cs. FIG. 7B is a sectional view of the motor shaft Ma and the tractor support shaft 21cs. FIG. 7C is an exploded perspective view of the motor shaft Ma and the tractor support shaft 21cs.

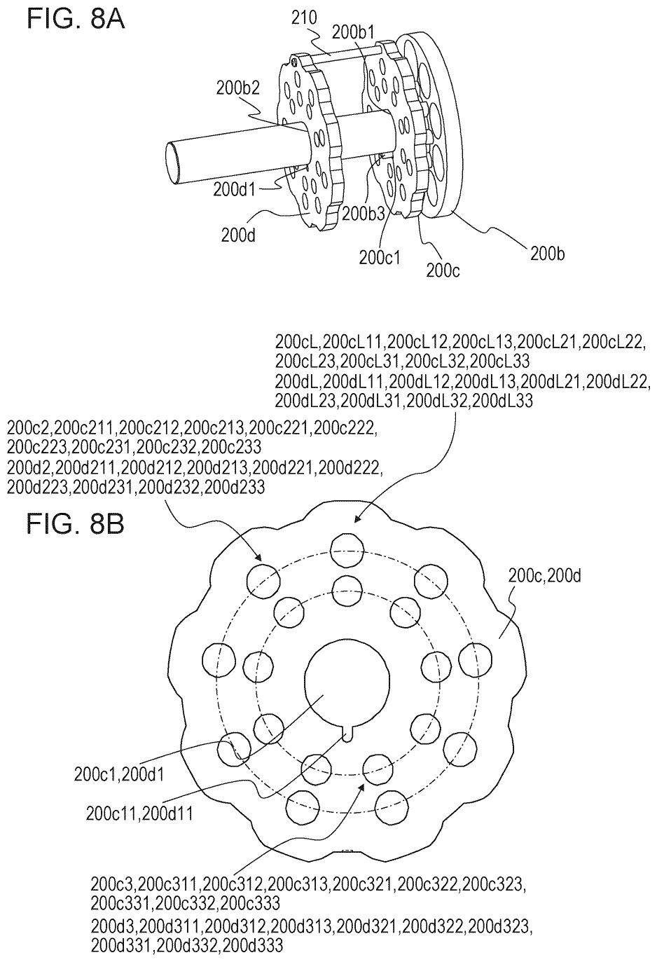

FIGS. 8A and 8B are views that illustrate the first bearing frame 200c and the second bearing frame 200d. FIG. 8A is a perspective view that shows a state where the first bearing frame 200c and the second bearing frame 200d are attached to the main frame 200b. FIG. 8B is a front view of the first bearing frame 200c and the second bearing frame 200d. FIG. 8B is a view when the first bearing frame 200c and the second bearing frame 200d are viewed in the rotational axis direction of the motor shaft Ma.

As shown in FIG. 7A, the first bearing frame (first supporting member) 200c and the second bearing frame (second supporting member) 200d are engaged with the main frame (coupling member) 200b and fixed. The motor shaft Ma is supported by the first bearing frame 200c via a drive bearing C1 and is supported by the second bearing frame 200d via a drive bearing C2. The tractor support shaft 21cs is supported by the first bearing frame 200c via the first bearing B1 and is supported by the second bearing frame 200d via the second bearing B2.

As shown in FIG. 8B, the first bearing frame 200c has a first engaging portion 200c1 that is engaged with the main frame 200b. In the present embodiment, the first engaging portion 200c1 is a hole. The first bearing frame 200c has first supporting portions (first supporting holes) 200c2 that each support the motor shaft Ma via the drive bearing C1. The first bearing frame 200c has second supporting portions (second supporting holes) 200c3 that each support the tractor support shaft 21cs via the first bearing B1.

The plurality of first supporting portions 200c2 is provided respectively in correspondence with the first to ninth driving sources (M11 to M33). In the present embodiment, the nine first supporting portions 200c2 (200c211, 200c212, 200c213, 200c221, 200c222, 200c223, 200c231, 200c232, 200c233) are provided. The last two digits of each reference sign indicates a corresponding driving source of the first to ninth driving sources (M11 to M33). The first supporting portions (200c211 to 200c233) are disposed along an imaginary circle with a predetermined radius so as to surround the first engaging portion 200c1.

The plurality of second supporting portions 200c3 is provided respectively in correspondence with the first to ninth coupling portions (21c11 to 21c33). In the present embodiment, the nine second supporting portions 200c3 (200c311, 200c312, 200c313, 200c321, 200c322, 200c323, 200c331, 200c332, 200c333) are provided. The last two digits of each reference sign indicates a corresponding coupling portion of the first to ninth coupling portions (21c11 to 21c33). The second supporting portions (200c311 to 200c333) are disposed along an imaginary circle with a predetermined radius so as to surround the first engaging portion 200c1.

When viewed in the rotational axis direction of the motor shaft Ma, a straight line that connects the center of each of the first supporting portions (200c211 to 200c233) with the center of a corresponding one of the second supporting portions (200c311 to 200c333) is referred to as first straight line. The first straight lines (200c L11, 200cL12, 200cL13, 200cL21, 200cL22, 200cL23, 200cL31, 200cL32, 200cL33) intersect with the first engaging portion 200c1. In the present embodiment, the first straight lines (200cL11 to 200cL33) intersect with a central axis 200x (described later).

As shown in FIG. 8B, the second bearing frame 200d has a second engaging portion 200d1 that is engaged with the main frame 200b. In the present embodiment, the second engaging portion 200d1 is a hole. The second bearing frame 200d has third supporting portions (third supporting holes) 200d2 that support the motor shaft Ma via the drive bearing C2. The second bearing frame 200d has fourth supporting portions (fourth supporting holes) 200d3 that each support the tractor support shaft 21cs via the second bearing B2.

The plurality of third supporting portions 200d2 is provided respectively in correspondence with the first to ninth driving sources (M11 to M33). In the present embodiment, the nine third supporting portions 200d2 (200d211, 200d212, 200d213, 200d221, 200d222, 200d223, 200d231, 200d232, 200d233) are provided. The last two digits of each reference sign indicates a corresponding driving source of the first to ninth driving sources (M11 to M33). The third supporting portions (200d211 to 200d233) are disposed along an imaginary circle with a predetermined radius so as to surround the second engaging portion 200d1.

The plurality of fourth supporting portions 200d3 is provided respectively in correspondence with the first to ninth coupling portions (21d11 to 21d33). In the present embodiment, the nine fourth supporting portions 200d3 (200d311, 200d312, 200d313, 200d321, 200d322, 200d323, 200d331, 200d332, 200d333) are provided. The last two digits of each reference sign indicates a corresponding coupling portion of the first to ninth coupling portions (21d11 to 21d33). The fourth supporting portions (200d311 to 200d333) are disposed along an imaginary circle with a predetermined radius so as to surround the second engaging portion 200d1.

When viewed in the rotational axis direction of the motor shaft Ma, a straight line that connects the center of each of the third supporting portions (200d211 to 200d233) with the center of a corresponding one of the fourth supporting portions (200d311 to 200d333) is referred to as second straight line. The second straight lines (200dL11, 200dL12, 200dL13, 200dL21, 200dL22, 200dL23, 200dL31, 200dL32, 200dL33) intersect with the second engaging portion 200d1. In the present embodiment, the second straight lines (200dL11 to 200dL33) intersect with the central axis 200x (described later).

In the direction of each of the first straight lines (200cL11 to 200cL33), each of the second supporting portions (200c311 to 200c333) is located between the first engaging portion 200c1 and a corresponding one of the first supporting portions (200c211 to 200c233). In the direction of each of the second straight lines (200dL11 to 200dL33), each of the fourth supporting portions (200d311 to 200d333) is located between the second engaging portion 200d1 and a corresponding one of the third supporting portions (200d211 to 200d233). As a result, the medical device 1 can be disposed so as to gradually narrow from each of the first to ninth driving sources (M11 to M33) toward the catheter 11, and the size of the medical device 1 is reduced.

The second bearing frame 200d has the same shape as the first bearing frame 200c. The second engaging portion 200d1 corresponds to the first engaging portion 200c1. The third supporting portions 200d2 correspond to the first supporting portions 200c2. The fourth supporting portions 200d3 correspond to the second supporting portions 200c3. The same shape here is achieved when parts related to supporting the motor shaft Ma and the tractor support shaft 21cs have the same shape. Specifically, this means that the first engaging portion 200c1 and the second engaging portion 200d1 have the same shape, the first supporting portions 200c2 and the third supporting portions 200d2 have the same shape, and the second supporting portions 200c3 and the fourth supporting portions 200d3 have the same shape. This also means that the positional relationship among the first engaging portion 200c1, the first supporting portions 200c2, and the second supporting portions 200c3 is the same as the positional relationship among the second engaging portion 200d1, the third supporting portions 200d2, and the fourth supporting portions 200d3.

In the rotational axis direction of the motor shaft Ma, the second bearing frame 200d is disposed at a location away from the first bearing frame 200c and is opposed to the first bearing frame 200c. The distance between the first bearing frame 200c and the second bearing frame 200d is maintained by the drive bearing C1, the drive bearing C2, and the motor shaft Ma.

As described above, the tractor 21ct and the tractor support shaft 21cs move in the direction of the rotational axis of the motor shaft Ma with rotation of the motor shaft Ma. To accurately move the first to ninth drive wires (W11 to W33), the motor shaft Ma and the tractor support shaft 21cs are preferably parallel to each other. When the motor shaft Ma and the tractor support shaft 21cs are not parallel to each other, sliding resistance at the time of converting rotational motion transmitted from the driving source M to linear motion of the tractor support shaft 21cs via the tractor 21ct changes. As a result, it is not possible to accurately move the tractor 21ct.

In the present embodiment, the first bearing frame 200c and the second bearing frame 200d having the same shape are engaged with the one main frame 200b. As a result, it is possible to suppress alignment deviation of the motor shaft Ma and the tractor support shaft 21cs between the first bearing frame 200c and the second bearing frame 200d.

Each of the direction of the imaginary circle along which the first supporting portions (200c211 to 200c233) are disposed, the direction of the imaginary circle along which the second supporting portions (200c311 to 200c333) are disposed, the direction of the imaginary circle along which the third supporting portions (200d211 to 200d233) are disposed, and the direction of the imaginary circle along which the fourth supporting portions (200d311 to 200d333) are disposed is referred to as circumferential direction. To bring the motor shaft Ma and the tractor support shaft 21cs close to an ideal parallel relationship, the phase of the first bearing frame 200c with respect to the main frame 200b and the phase of the third bearing frame 200d with respect to the main frame 200b are preferably the same in the circumferential direction.

A straight line that connects the center of the imaginary circle along which the first supporting portions (200c211 to 200c233) are disposed with the center of the imaginary circle along which the third supporting portions (200d211 to 200d233) are disposed may be referred to as central axis 200x. A straight line that connects the center of the imaginary circle along which the second supporting portions (200c311 to 200c333) are disposed with the center of the imaginary circle along which the fourth supporting portions (200d311 to 200d333) are disposed also coincides with the central axis 200x. In the present embodiment, a central axis of a cylindrical part of the main frame 200b inserted through the first engaging portion 200c1 and the second engaging portion 200d1 also coincides with the central axis 200x.

In order to accurately fix the first bearing frame 200c and the second bearing frame 200d to the main frame 200b, the radius of the first engaging portion 200c1, the radius of the second engaging portion 200d1, and the radius of the cylindrical part of the main frame 200b are preferably large. In the present embodiment, the radius of the first engaging portion 200c1 is greater than the radius of each of the first supporting portions (200c211 to 200c233) and is also greater than the radius of each of the second supporting portions (200c311 to 200c333). The radius of the second engaging portion 200d1 is greater than the radius of each of the third supporting portions (200d211 to 200d233) and is also greater than the radius of each of the fourth supporting portions (200d311 to 200d333).

As shown in FIGS. 7A, 7B, and 7C, the main frame 200b has a rib (rotation regulating portion) 200b3 parallel to the central axis 200x of the main frame 200b. On the other hand, as shown in FIG. 8B, the first engaging portion 200c1 and the second engaging portion 200d1 respectively have grooves (rotation regulated portions) (200c11, 200d11) that are engaged with the rib 200b3.

The first engaging portion 200c1 of the first bearing frame 200c and the second engaging portion 200d1 of the second bearing frame 200d are respectively engaged with supporting portions 200b1, 200b2 of the main frame 200b. At this time, the rib 200b3 is engaged with the grooves (200c11, 200d11). Thus, the phase of the first bearing frame 200c and the phase of the second bearing frame 200d are in the same phase in the circumferential direction. The groove (200c11) extends toward a part between the adjacent second supporting portions 200c3. The groove (200d11) extends toward a part between the adjacent fourth supporting portions 200d3. Therefore, the second supporting portions 200c3 can be disposed close to the first engaging portion 200c1, and the fourth supporting portions 200d3 can be disposed close to the second engaging portion 200d1.

As shown in FIG. 8A, the rib 200b3 of the main frame 200b may be engaged with the groove 200c11 of the first bearing frame 200c, and the second bearing frame 200d may be fixed in phase with the first bearing frame 200c by using a metal shaft 210. The number of the metal shafts 210 is one in FIG. 8A. Alternatively, a plurality of the metal shafts 210 may be used.

The rib 200b3, the grooves (200c11, 200d11), and the metal shaft 210 function as a displacement regulating portion (rotation regulating portion) that regulates displacement (rotation) of the second bearing frame 200d with respect to the first bearing frame 200c in the circumferential direction of the above-described imaginary circle.

A step of assembling the motor shaft Ma, the tractor support shaft 21cs, the first bearing frame 200c, the second bearing frame 200d, and the main frame 200b will be described.

In the present embodiment, the number of the motor shafts Ma and the number of the tractor support shafts 21cs each are nine, a method of assembling the motor shaft Ma is the same and a method of assembling the tractor support shaft 21cs is the same. Therefore, here, assembling of one motor shaft Ma and one tractor support shaft 21cs will be described.

Initially, the first engaging portion 200c1 of the first bearing frame 200c is engaged with the main frame 200b. The motor shaft Ma is attached to the first supporting portion 200c2 of the first bearing frame 200c via the drive bearing C1. The tractor 21ct is coupled to the motor shaft Ma. The second engaging portion 200d1 of the second bearing frame 200d is engaged with the main frame 200b. The motor shaft Ma is attached to the third supporting portion 200d2 of the second bearing frame 200d via the drive bearing C2. As a result, the motor shaft Ma is supported by the first supporting portion 200c2 of the first bearing frame 200c via the drive bearing C1 and is supported by the third supporting portion 200d2 of the second bearing frame 200d via the drive bearing C2.

Furthermore, the tractor support shaft 21cs and the tractor 21ct are coupled. The tractor support shaft 21cs is attached to the second supporting portion 200c3 of the first bearing frame 200c via the first bearing B1 and is attached to the fourth supporting portion 200d4 of the second bearing frame 200d via the second bearing B2. As a result, the tractor support shaft 21cs is supported by the second supporting portion 200c3 of the first bearing frame 200c via the first bearing B1 and the fourth supporting portion 200d3 of the second bearing frame 200d via the second bearing B2.

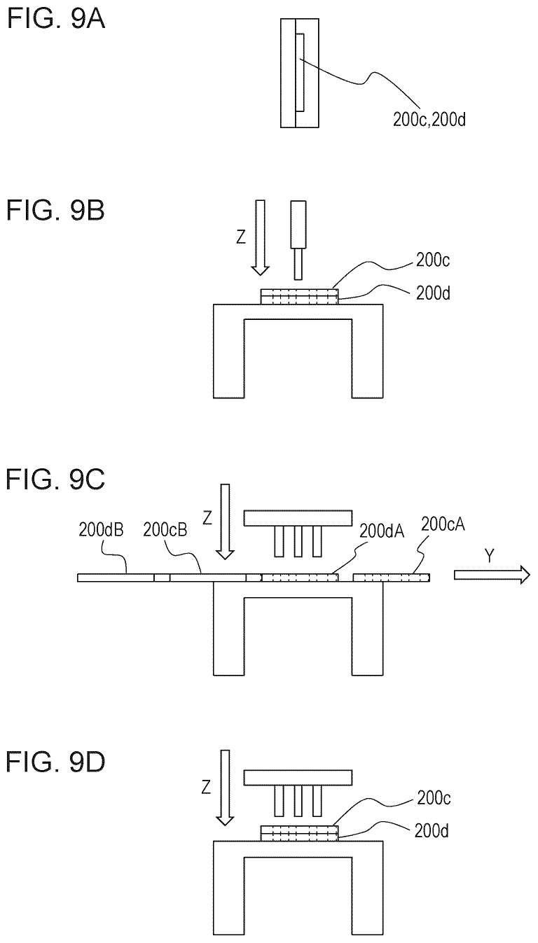

A manufacturing method for the base unit 200 includes a step of manufacturing and preparing the first bearing frame 200c and the second bearing frame 200d. FIGS. 9A to 9D are views that illustrate a step of manufacturing the first bearing frame 200c and the second bearing frame 200d.

Hereinafter, a preferred manufacturing method in manufacturing the first bearing frame 200c and the second bearing frame 200d that are used in one base unit 200 will be described.

FIG. 9A is a view that illustrates a step of manufacturing the first bearing frame 200c and the second bearing frame 200d by injection molding using the same die. FIG. 9B is a view that illustrates a step of manufacturing the first bearing frame 200c and the second bearing frame 200d by removal processing. FIGS. 9C and 9D are views that illustrate a step of manufacturing the first bearing frame 200c and the second bearing frame 200d by press working using the same die.

As shown in FIG. 9A, when the first bearing frame 200c and the second bearing frame 200d are manufactured by injection molding using the same die, the first bearing frame 200c and the second bearing frame 200d having the same shape are manufactured.

A material at the time of performing injection molding of the first bearing frame 200c and the second bearing frame 200d in the present embodiment may be a resin, a metal, such as aluminum, or a mixed material of them.

As shown in FIG. 9B, the first bearing frame 200c and the second bearing frame 200d may be manufactured by removal processing. Specifically, when part of a plurality of materials is removed in a state where the plurality of materials is stacked, the first bearing frame 200c and the second bearing frame 200d can be manufactured. As shown in FIG. 9B, cutting may be used as an example of a method of removing part of a plurality of materials. In cutting, a cutting tool movable in a direction of an arrow Z and in a direction orthogonal to the arrow Z is used.

As shown in FIG. 9B, when a plurality of materials is subjected to cutting in a state of being stacked, the first bearing frame 200c and the second bearing frame 200d can be manufactured in a state where the degree of abrasion and temperature of a processing apparatus remain unchanged. As a result, the first bearing frame 200c and the second bearing frame 200d having the same shape are manufactured.

Since the base unit 200 includes the pair of first bearing frame 200c and second bearing frame 200d, the number of materials to be stacked is preferably an even number. The number of materials to be stacked may be other than two, and may be four, six, or the like.

As shown in FIGS. 9C and 9D, the first bearing frame 200c and the second bearing frame 200d may be manufactured by press working using the same die. In press working, a press tool (die) movable in the direction of the arrow Z is used.

As shown in FIG. 9C, while a sheet-shaped material is being moved in an arrow Y direction, part of the material is removed by press working, and the first bearing frame 200c and the second bearing frame 200d are alternately manufactured.

The first bearing frame 200c and the second bearing frame 200d that are used in one base unit 200 are preferably the first bearing frame 200c and the second bearing frame 200d that are adjacent in order of manufacturing. For example, a first bearing frame 200cA and a second bearing frame 200dA manufactured subsequently to the first bearing frame 200cA are preferably used in one base unit 200. Furthermore, a first bearing frame 200cB manufactured subsequently to the second bearing frame 200dA and a second bearing frame 200dB manufactured subsequently to the first bearing frame 200cB are preferably used in another base unit 200.

In this way, when the first bearing frame 200c and the second bearing frame 200d are alternately manufactured by press working using the same die, the first bearing frame 200c and the second bearing frame 200d can be manufactured in a state where the degree of abrasion and temperature of a processing apparatus remain unchanged. As a result, the first bearing frame 200c and the second bearing frame 200d having the same shape are manufactured.

As shown in FIG. 9D, press working may be used as an example of a method of removing part of a plurality of materials. As shown in FIG. 9B, when a plurality of materials is subjected to press working in a state of being stacked, the first bearing frame 200c and the second bearing frame 200d can be manufactured in a state where the degree of abrasion and temperature of a processing apparatus remain unchanged. As a result, the first bearing frame 200c and the second bearing frame 200d having the same shape are manufactured.

Since the base unit 200 includes the pair of first bearing frame 200c and second bearing frame 200d, the number of materials to be stacked is preferably an even number. The number of materials to be stacked may be other than two, and may be four, six, or the like.

As described above, the first bearing frame 200c and the second bearing frame 200d that are used in one base unit 200 can be manufactured so as to have the same shape.

As a result, the positional relationship among the first engaging portion 200c1, the first supporting portions 200c2, and the second supporting portions 200c3 is the same as the positional relationship among the second engaging portion 200d1, the third supporting portions 200d2, and the fourth supporting portions 200d3. The first engaging portion 200c1 and the second engaging portion 200d1 have the same shape. The first supporting portions 200c2 and the third supporting portions 200d2 have the same shape. The second supporting portions 200c3 and the fourth supporting portions 200d3 have the same shape. Furthermore, the phase of the first bearing frame 200c with respect to the main frame 200b and the phase of the second bearing frame 200d with respect to the main frame 200b are preferably the same in the circumferential direction. As a result, a deviation of alignment between the motor shaft Ma and the tractor support shaft 21cs is suppressed, so the motor shaft Ma and the tractor support shaft 21cs are kept in a state close to a geometrical parallel relation.

When the first bearing frame 200c and the second bearing frame 200d cannot be formed completely in the same shape, the first bearing frame 200c and the second bearing frame 200d may be formed with the above-described methods and then formed as another shape by adding another step. For example, in a range in which the positional relationship among the first bearing frame 200c, the second bearing frame 200d, and the main frame 200b remains unchanged, the shape of the first engaging portion 200c1 and the shape of the second engaging portion 200d1 may be different from each other. In a range in which the positional relationship among the first bearing frame 200c, the second bearing frame 200d, and the motor shaft Ma remains unchanged, the shape of each first supporting portion 200c2 and the shape of each third supporting portion 200d2 may be different from each other. In a range in which the positional relationship among the first bearing frame 200c, the second bearing frame 200d, and the tractor support shaft 200cs remains unchanged, the shape of each second supporting portion 200c3 and the shape of each fourth supporting portion 200d3 may be different from each other. The shape of the first bearing frame 200c and the shape of the second bearing frame 200d may be different from each other between portions other than between the first engaging portion 200c1 and the second engaging portion 200d1, between the first supporting portions 200c2 and the third supporting portions 200d2, or between the second supporting portions 200c3 and the fourth supporting portions 200d3.

With the above-described base unit 200, the motor shaft Ma and the tractor support shaft 21cs can be accurately positioned. As a result, a deviation of alignment between the motor shaft Ma and the tractor support shaft 21cs is suppressed, so the motor shaft Ma and the tractor support shaft 21cs are kept in a state close to a geometrical parallel relation.

Therefore, it is possible to accurately convert the rotational motion of the driving source M to linear motion, so it is possible to accurately move the tractor 21ct. For this reason, it is possible to accurately move the drive wire W and accurately, stably bend the bend portion 12. When a user replaces the catheter unit 100 as well, it is possible to accurately, stably bend the bend portion 12.

### Attachment of Catheter Unit

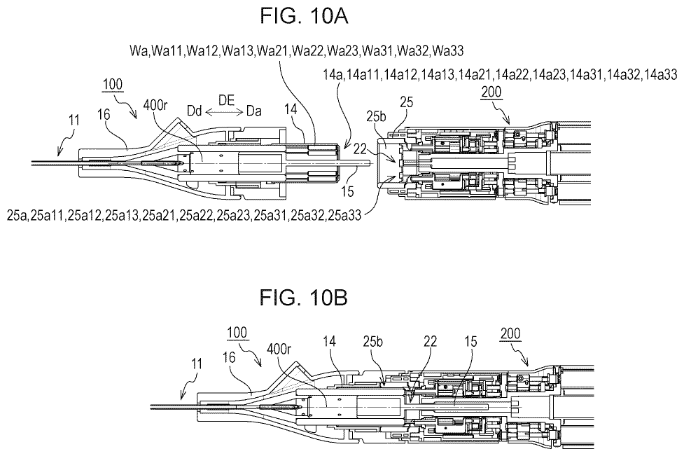

An operation to attach the catheter unit 100 to the base unit 200 will be described with reference to FIGS. 10A and 10B.

FIGS. 10A and 10B are views that illustrate attachment of the catheter unit 100. FIG. 10A is a view before the catheter unit 100 is attached to the base unit 200. FIG. 10B is a view after the catheter unit 100 is attached to the base unit 200.

In the present embodiment, the attaching and detaching direction DE of the catheter unit 100 is the same as the direction of the rotation axis 400r of the operating portion 400. In the attaching and detaching direction DE, the direction in which the catheter unit 100 is attached to the base unit 200 is referred to as the attachment direction Da. In the attaching and detaching direction DE, the direction in which the catheter unit 100 is detached from the base unit 200 (a direction opposite to the attachment direction Da) is referred to as a detachment direction Dd.

As shown in FIG. 10A, in a state before the catheter unit 100 is attached to the base unit 200, the wire cover 14 is placed at the cover position. At this time, the wire cover 14 covers the first to ninth drive wires (W11 to W33) such that the first to ninth held portions (Wa11 to Wa33) do not protrude from the first to ninth exposure holes (14a11 to 14a33) of the wire cover 14. Therefore, in a state before the catheter unit 100 is attached to the base unit 200, the first to ninth drive wires (W11 to W33) are protected.

When the catheter unit 100 is attached to the base unit 200, the key shaft 15 is engaged with the key receiving portion 22. The key shaft 15 protrudes from the wire cover 14. In the present embodiment, in a state where the key shaft 15 has reached the entrance of the key receiving portion 22, the wire cover 14 is not engaged with the attachment opening 25b. In other words, when the phase of the catheter unit 100 with respect to the base unit 200 is a phase in which the key shaft 15 and the key receiving portion 22 are not engaged with each other, the wire cover 14 is not engaged with the attachment opening 25b, and the state where the wire cover 14 is placed at the cover position is maintained. Therefore, even when the catheter unit 100 is moved such that the key shaft 15 and the key receiving portion 22 are engaged with each other, the first to ninth drive wires (W11 to W33) are protected.

When the key shaft 15 and the key receiving portion 22 are engaged with each other and the catheter unit 100 is moved in the attachment direction Da with respect to the base unit 200, the catheter unit 100 is attached to the base unit 200. When the catheter unit 100 is attached to the base unit 200, the wire cover 14 is moved to the exposed position. In the present embodiment, the wire cover 14 contacts with the base frame 25 to move from the cover position to the exposed position (see FIG. 10B).

More specifically, when the catheter unit 100 is attached, the wire cover 14 contacts with the base frame 25 to stop. In this state, by moving the catheter unit 100 in the attachment direction Da, the wire cover 14 relatively moves with respect to a part other than the wire cover 14 in the catheter unit 100. As a result, the wire cover 14 moves from the cover position to the exposed position.

The wire cover 14 moves from the cover position to the exposed position, while the held portion Wa of the drive wire W protrudes from the exposure hole 14a of the wire cover 14 and is inserted into the insertion hole 25a. Then, the held portion Wa is engaged with the leaf spring 21ch of the coupling portion 21c (see FIG. 6B).

In a state where the catheter unit 100 is just attached to the base unit 200, the catheter unit 100 can be detached by moving the catheter unit 100 in the detachment direction Dd with respect to the base unit 200. As will be described later, in a state where the catheter unit 100 is just attached to the base unit 200, fixing of the drive wire W with the coupling portion 21c is in an unlocked state.

In a state where the catheter unit 100 is attached to the base unit 200, detachment of the catheter unit 100 from the base unit 200 by operating the operating portion 400 is prevented. In addition, by operating the operating portion 400 in a state where the catheter unit 100 is attached to the base unit 200, the bend drive portion 13 is fixed to the coupling device 21, and the bend drive portion 13 is coupled to the wire drive portion 300 via the coupling device 21.

### Fixing of Bend Drive Portion and Unlocking of Fixing

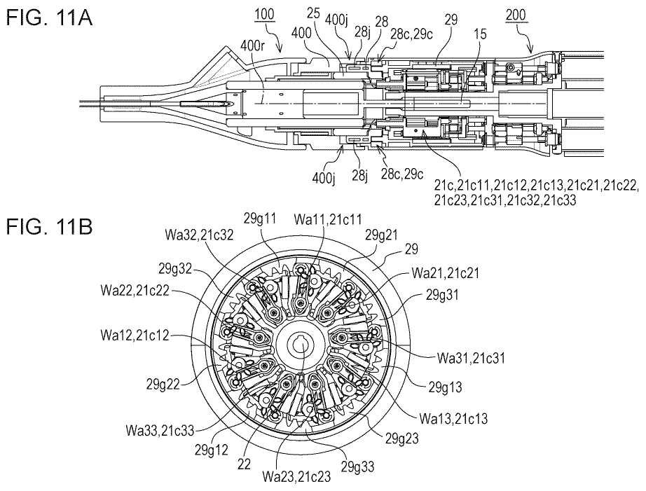

A configuration for fixing the bend drive portion 13 to the coupling device 21 and a configuration for unlocking fixing of the bend drive portion 13 with the coupling device 21 will be described with reference to FIGS. 11A, 11B, 12, 13, 14, 15, 16, and 17.

FIGS. 11A and 11B are views that illustrate coupling of the catheter unit 100 with the base unit 200. FIG. 11A is a sectional view of the catheter unit 100 and the base unit 200. FIG. 11A is a sectional view of the catheter unit 100 and the base unit 200, taken along the rotation axis 400r. FIG. 11B is a sectional view of the base unit 200. FIG. 11B is a sectional view of the base unit 200, taken in a direction orthogonal to the rotation axis 400r at a part of the coupling portion 21c.

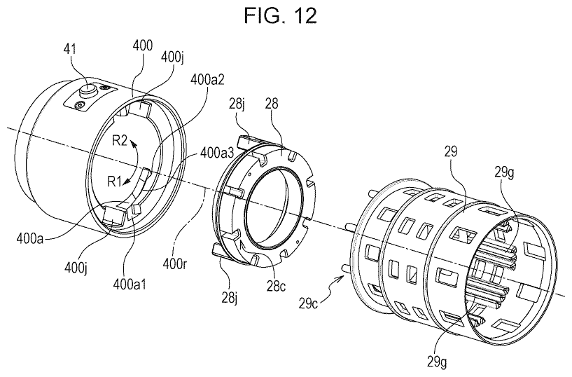

FIG. 12 is an exploded view that illustrates coupling of the catheter unit 100 with the base unit 200.

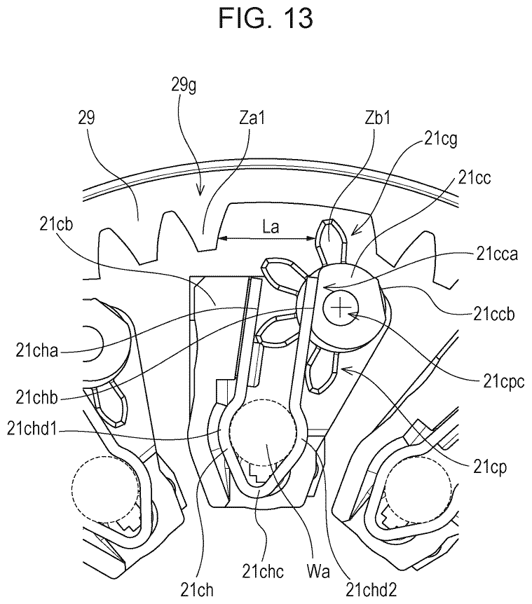

FIGS. 13, 14, 15, 16, and 17 are views that illustrate fixing of the drive wire W with the coupling portion 21c.

As shown in FIGS. 11A and 12, the base unit 200 includes the joint (an intermediate member or a second transmission member) 28, and the internal gear 29 serving as a movable gear (an interlocking gear, a transmission member, or a first transmission member) that is in interlocking with the operating portion 400 via the joint 28.

The joint 28 has a plurality of transmitting portions 28c. The internal gear 29 has a plurality of transmitted portions 29c. The plurality of transmitting portions 28c is engaged with the plurality of transmitted portions 29c, and, when the joint 28 rotates, rotation of the joint 28 is transmitted to the internal gear 29.

When the catheter unit 100 is attached to the base unit 200, the engagement portion 400j provided in the operating portion 400 is engaged with the joint engagement portion 28j of the joint 28. When the operating portion 400 rotates, rotation of the operating portion 400 is transmitted to the joint 28. The operating portion 400, the joint 28, and the internal gear 29 rotate in the same direction.

The internal gear 29 has a plurality of tooth portions for switching between a state where each of the first to ninth coupling portions (21c11 to 21c33) fixes a corresponding one of the first to ninth drive wires (W11 to W33) and a state where each of the first to ninth coupling portions (21c11 to 21c33) releases a corresponding one of the first to ninth drive wires (W11 to W33). Each of the plurality of tooth portions (working portions or gear switching portions) of the internal gear 29 is engaged with the gear portion 21cg of the pressing member 21cp of each of the first to ninth coupling portions (21c11 to 21c33).

Specifically, in the present embodiment, the internal gear 29 has the first tooth portion 29g11, the second tooth portion 29g12, the third tooth portion 29g13, the fourth tooth portion 29g21, the fifth tooth portion 29g22, the sixth tooth portion 29g23, the seventh tooth portion 29g31, the eighth tooth portion 29g32, and the ninth tooth portion 29g33. The first to ninth tooth portions (29g11 to 29g33) are formed with a gap from each other.

The first tooth portion 29g11 meshes with the gear portion 21cg of the first coupling portion 21c11. The second tooth portion 29g12 meshes with the gear portion 21cg of the second coupling portion 21c12. The third tooth portion 29g13 meshes with the gear portion 21cg of the third coupling portion 21c13. The fourth tooth portion 29g21 meshes with the gear portion 21cg of the fourth coupling portion 21c21. The fifth tooth portion 29g22 meshes with the gear portion 21cg of the fifth coupling portion 21c22. The sixth tooth portion 29g23 meshes with the gear portion 21cg of the sixth coupling portion 21c23. The seventh tooth portion 29g31 meshes with the gear portion 21cg of the seventh coupling portion 21c31. The eighth tooth portion 29g32 meshes with the gear portion 21cg of the eighth coupling portion 21c32. The ninth tooth portion 29g33 meshes with the gear portion 21cg of the ninth coupling portion 21c33.

Of the first to ninth tooth portions (29g11 to 29g33), a selected one may be referred to as a tooth portion 29g. In the present embodiment, each of the first to ninth tooth portions (29g11 to 29g33) has the same configuration.

In the present embodiment, the configuration in which each of the first to ninth drive wires (W11 to W33) is coupled to a corresponding one of the first to ninth coupling portions (21c11 to 21c33) is the same. The configuration in which each of the first to ninth coupling portions (21c11 to 21c33) is connected to a corresponding one of the first to ninth tooth portions (29g11 to 29g33) is the same. Therefore, in the following description, the configuration in which the drive wire W, the coupling portion 21c, and the tooth portion 29g are connected will be described by using the one drive wire W, the one coupling portion 21c, and the one tooth portion 29g.

In each of the first to ninth coupling portions (21c11 to 21c33), when the gear portion 21cg is moved by the internal gear 29, the pressing member 21cp rotates, and the cam 21cc moves to a pressing position or to a retracted position retracted from the pressing position.

By rotating the operating portion 400, the internal gear 29 rotates. When the internal gear 29 rotates, each of the first to ninth coupling portions (21c11 to 21c33) operates. In other words, with an operation to rotate the one operating portion 400, the first to ninth coupling portions (21c11 to 21c33) are actuated.

The operating portion 400 is allowed to move to a fixed position (lock position) and a detachment position in a state where the catheter unit 100 is attached to the base unit 200. As will be described later, the operating portion 400 is allowed to move to the unlock position in a state where the catheter unit 100 is attached to the base unit 200. In the circumferential direction of the operating portion 400, the unlock position is located between the fixed position and the detachment position. In a state where the operating portion 400 is placed at the detachment position, the catheter unit 100 is attached to the base unit 200.

In a state where the catheter unit 100 is attached to the base unit 200, fixing (locking) of the drive wire W to the coupling portion 21c is unlocked. This state is referred to as an unlocked state of the coupling portion 21c. A state where the drive wire W is fixed (locked) to the coupling portion 21c is referred to as a locked state of the coupling portion 21c.

An operation at the time of fixing the drive wire W to the coupling portion 21c will be described with reference to FIGS. 13, 14, 15, 16, and 17.

In a state after the catheter unit 100 is attached to the base unit 200 and before the operating portion 400 is operated, the catheter unit 100 is allowed to be detached from the base unit 200. Hereinafter, a state where the catheter unit 100 is allowed to be detached from the base unit 200 is referred to as a detachable state.

FIG. 13 is a view that shows a state of the internal gear 29 and the coupling portion 21c in the detachable state. FIG. 13 is a view that shows the internal gear 29 and the coupling portion 21c in a state where the operating portion 400 is placed at the detachment position.

The leaf spring 21ch of the coupling portion 21c has a fixed portion 21cha fixed to the coupling base 21cb, and a pressed portion 21chb that contacts with the cam 21cc of the pressing member 21cp. The leaf spring 21ch has a first part 21chd1 and a second part 21chd2. When the catheter unit 100 is attached to the base unit 200, the held portion Wa is inserted between the first part 21chd1 and the second part chd2.

The cam 21cc has a holding surface 21cca and a pressing surface 21ccb. In a radial direction of rotation of the pressing member 21cp, the holding surface 21cca is disposed at a position closer to a rotation center 21cpc of the pressing member 21cp than the pressing surface 21ccb.

As shown in FIG. 13, in the detachable state (a state where the operating portion 400 is at the detachment position), the leaf spring 21ch is held at a position at which the pressed portion 21chb is in contact with the holding surface 21cca. A tooth Za1 of the internal gear 29 and a tooth Zb1 of the gear portion 21cg are stopped in a state where there is a clearance La therebetween.

In the rotation direction of the operating portion 400, a direction in which the operating portion 400 heads from the detachment position for the unlock position and the fixed position is referred to as a lock direction (fixing direction), and a direction in which the operating portion 400 heads from the fixed position for the unlock position and the detachment position is referred to as an unlock direction. The operating portion 400 rotates in the unlock direction from the unlock position and moves to the detachment position. The operating portion 400 rotates in the lock direction from the unlock position to move to the fixed position.

In a state where the catheter unit 100 is attached to the base unit 200 and the operating portion 400 is at the detachment position, the coupling portion 21c is in an unlocked state, and fixing of the drive wire W with the coupling portion 21c is unlocked.

When the coupling portion 21c is in the unlocked state, the cam 21cc is placed at the retracted position retracted from the pressing position (described later). At this time, fixing of the held portion Wa with the leaf spring 21ch is unlocked. A force that the first part 21chd1 and the second part 21chd2 fasten the held portion Wa when the coupling portion 21c is in the unlocked state is less than a force that the first part 21chd1 and the second part 21chd2 fasten the held portion Wa when the coupling portion 21c is in the locked state.

When the catheter unit is moved in the detachment direction Dd with respect to the base unit 200 while the coupling portion 21c is in the unlocked state, the held portion Wa can be pulled out from between the first part 21chd1 and the second part 21chd2.

It is desirable that no force that the first part 21chd1 and the second part 21chd2 fasten the held portion Wa be generated (a state where the magnitude is zero) when the coupling portion 21c is in the unlocked state. It is desirable that a clearance be formed between the held portion Wa and at least any one of the first part 21chd1 and the second part 21chd2 when the coupling portion 21c is in the unlocked state.

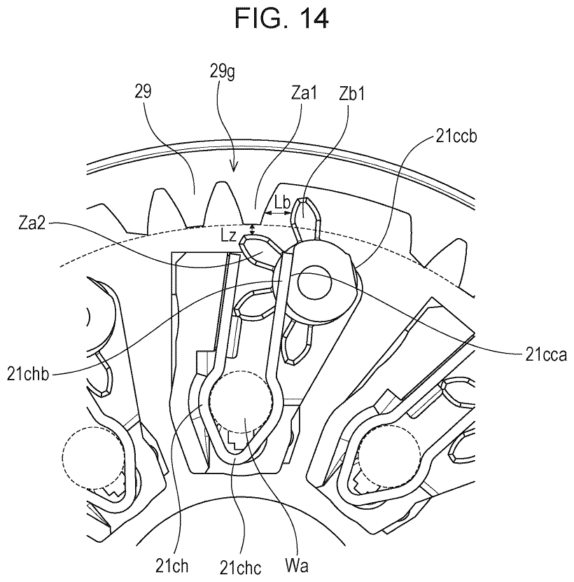

FIG. 14 is a view that shows a state of the internal gear 29 and the coupling portion 21c when the operating portion 400 is rotated in the lock direction from the detachment position. FIG. 14 is a view that shows a state of the internal gear 29 and the coupling portion 21c in a state where the operating portion 400 is at the unlock position.

When the operating portion 400 is rotated in the lock direction in a state where the operating portion 400 is at the detachment position (FIG. 13), the internal gear 29 rotates in the clockwise direction. Then, the operating portion 400 is placed at the unlock position.

Even when the operating portion 400 is rotated, the key shaft 15 and the key receiving portion 22 are engaged with each other, so rotation of the whole (except the operating portion 400) of the catheter unit 100 with respect to the base unit 200 is restricted. In other words, the operating portion 400 is rotatable with respect to the whole (except the operating portion 400) of the catheter unit 100 and the base unit 200 in a state where the whole (except the operating portion 400) of the catheter unit 100 and the base unit 200 are stopped.

When the internal gear 29 rotates in the clockwise direction, the clearance between the toothZa1 of the internal gear 29 and the tooth Zb1 of the gear portion 21cg reduces from a clearance La to a clearance Lb.

A tooth Zb2 of the gear portion 21cg is disposed at a position spaced a clearance Lz apart from a tip circle (dashed line) of the tooth portion 29g of the internal gear 29. Therefore, the internal gear 29 is rotatable without interference with the tooth Zb2. On the other hand, the coupling portion 21c is maintained in the same state (unlocked state) as the state shown in FIG. 13.

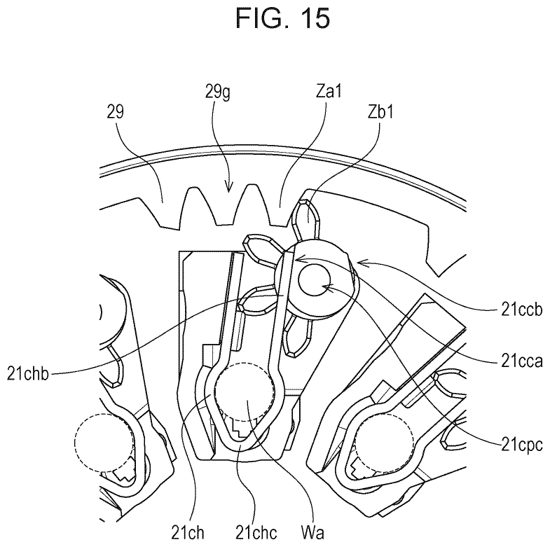

When the operating portion 400 is further rotated from the state shown in FIG. 14 in the lock direction, the internal gear 29 is further rotated in the clockwise direction. FIG. 15 shows a state of the internal gear 29 and the coupling portion 21c at that time.

FIG. 15 is a view that shows a state of the internal gear 29 and the coupling portion 21c when the operating portion 400 is rotated in the lock direction from the unlock position.

As shown in FIG. 15, when the operating portion 400 is rotated in the lock direction from the unlock position, the tooth Za1 of the internal gear 29 and the tooth Zb1 of the gear portion 21cg contact with each other. On the other hand, the coupling portion 21c is maintained in the unlocked state that is the same state as the state shown in FIGS. 13 and 14.

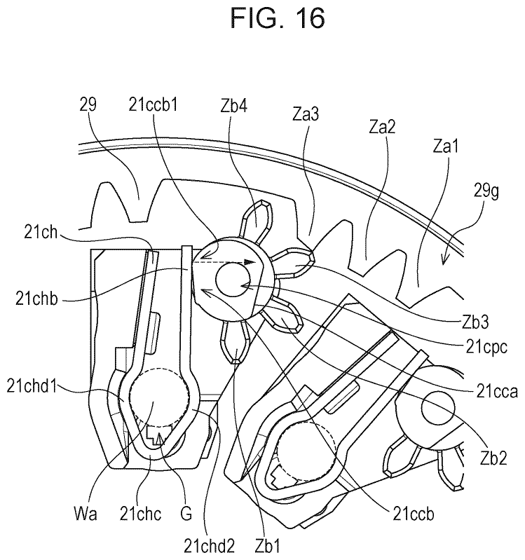

FIG. 16 is a view that shows a state where the pressing member 21cp is rotated as a result of rotation of the operating portion 400 in the lock direction.

As shown in FIG. 16, when the operating portion 400 is further rotated from the state shown in FIG. 16 in the lock direction, the internal gear 29 is further rotated in the clockwise direction.

The internal gear 29 shifts from the state of FIG. 15 to the state of FIG. 16, with the result that the internal gear 29 rotates the gear portion 21cg in the clockwise direction. When the gear portion 21cg rotates, the holding surface 21cca separates from the pressed portion 21chb, and the pressing surface 21ccb approaches the pressed portion 21chb. Then, the first part 21chd1 and the second part 21chd2 begin to clamp the held portion Wa.

Then, while the pressed portion 21chb is pressed by a corner 21ccb1 disposed at the end portion of the pressing surface 21ccb, a tooth Za3 of the internal gear 29 moves to a position separated from a tooth Zb3 of the gear portion 21cg. At this time, the held portion Wa is clamped by the first part 21chd1 and the second part 21chd2.

When the tooth Za3 of the internal gear 29 separates from the tooth Zb3 of the gear portion 21cg, transmission of driving force from the internal gear 29 to the gear portion 21cg stops. At this time, the corner 21ccb1 of the cam 21cc receives a reaction force from the leaf spring 21ch.

In the radial direction of rotation of the pressing member 21cp, the reaction force of the leaf spring 21ch, applied to the corner 21ccb1, is applied at a position spaced apart from the rotation center 21cpc of the pressing member 21cp, and the pressing member 21cp rotates in the clockwise direction. At this time, the pressing member 21cp rotates in the same direction as the direction to be rotated by the internal gear 29 that rotates in the clockwise direction.

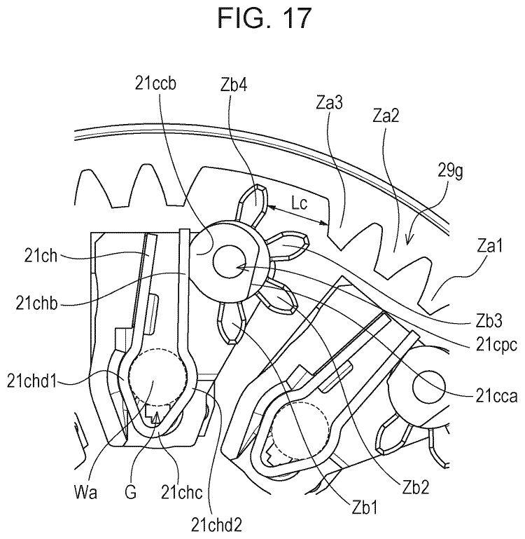

FIG. 17 is a view that shows a state of the internal gear 29 and the coupling portion 21c in a state where the operating portion 400 is at the fixed position.

As shown in FIG. 17, the pressing member 21cp further rotates from the state shown in FIG. 16 upon receiving the reaction force of the leaf spring 21ch.

As shown in FIG. 17, the pressing member 21cp stops in a state where the pressing surface 21ccb of the cam 21cc and the pressed portion 21chb of the leaf spring 21ch are in area contact with each other. In other words, the pressing surface 21ccb and the surface of the pressed portion 21chb are in a state of being arranged in the same plane.

At this time, the coupling portion 21c is in a locked state. When the coupling portion 21c is in the locked state, the cam 21cc of the pressing member 21cp is placed at the pressing position, and the pressing surface 21ccb presses the pressed portion 21chb.

When the coupling portion 21c is in the locked state, the held portion Wa is clamped by the first part 21chd1 and the second part 21chd2. In other words, the leaf spring 21ch is pressed by the cam 21cc, and the held portion Wa is fastened by the leaf spring 21ch. As a result, the held portion Wa is fixed by the leaf spring 21ch.

In the present embodiment, in the leaf spring 21ch, the first part 21chd1 and the second part 21chd2 press the held portion Wa at positions spaced apart from each other. In addition, a bent portion 21chc is disposed between the first part 21chd1 and the second part 21chd2 to connect the first part 21chd1 and the second part 21chd2. The bent portion 21chc is disposed with a gap G from the held portion Wa. Thus, the held portion Wa is stably fixed by the first part 21chd1 and the second part 21chd2.

The material of the leaf spring 21ch may be a resin or a metal and is preferably a metal.

When the coupling portion 21c is in the locked state, the held portion Wa is restricted from being pulled out from between the first part 21chd1 and the second part 21chd2.

The tooth Za3 of the internal gear 29 and a tooth Zb4 of the gear portion 21cg are stopped at positions where there is a clearance Lc therebetween.

When fixing of the drive wire W to the coupling portion 21c is unlocked, the operating portion 400 placed at the fixed position is rotated in the unlock direction. At this time, the internal gear 29 rotates from the state shown in FIG. 17 in the counterclockwise direction. When the internal gear 29 rotates in the counterclockwise direction, the tooth Za3 of the internal gear 29 contacts with the tooth Zb4 of the gear portion 21cg, and the pressing member 21cp is rotated in the counterclockwise direction.

By further rotating the internal gear 29 in the counterclockwise direction, fixing of the drive wire W with the coupling portion 21c is unlocked. The operations of the internal gear 29 and the pressing member 21cp at this time are operations reverse to the above-described operations. In other words, fixing of the drive wire W with the coupling portion 21c is unlocked by the operation reverse to the operation at the time of fixing the above-described drive wire W with the coupling portion 21c.

The above-described operations are performed in each of the first to ninth coupling portions (21c11 to 21c33). In other words, in the course in which the operating portion 400 moves from the detachment position to the fixed position, the first to ninth coupling portions (21c11 to 21c33) shift from the unlocked state to the locked state by movement (rotation) of the operating portion 400. In the course in which the operating portion 400 moves from the fixed position to the detachment position, the first to ninth coupling portions (21c11 to 21c33) shift from the locked state to the unlocked state by movement (rotation) of the operating portion 400. In other words, a user is able to switch the plurality of coupling portions between the unlocked state and the locked state by operating the single operating portion 400.

In other words, it is not necessary that each of the plurality of coupling portions includes an operating portion for switching between the unlocked state and the locked state and a user operates the plurality of coupling portions. Therefore, a user is able to easily attach and detach the catheter unit 100 to the base unit 200. In addition, the medical device 1 is simplified.

A state where each of the first to ninth drive wires (W11 to W33) is fixed by a corresponding one of the first to ninth coupling portions (21c11 to 21c33) is referred to as a first state. A state where fixing of each of the first to ninth drive wires (W11 to W33) with a corresponding one of the first to ninth coupling portions (21c11 to 21c33) is unlocked is referred to as a second state.

Interlocking with the movement of the operating portion 400, the state is switched between the first state and the second state. In other words, interlocking with the movement of the operating portion 400 between the detachment position and the fixed position, the state is switched between the first state and the second state.

The internal gear 29 is configured to interlock with the operating portion 400. In the present embodiment, the joint 28 functions as a transmission member for interlocking the operating portion 400 with the internal gear 29. The internal gear 29 and the joint 28 function as an interlocking portion that interlocks with the operating portion 400 such that the state switches between the first state and the second state interlocking with the movement of the operating portion 400.

Specifically, the internal gear 29 and the joint 28 move part (pressed portion 21chb) of the leaf spring 21ch toward the held portion Wa interlocking with the movement of the operating portion 400 in a state where the catheter unit 100 is attached to the base unit 200. When the held portion 21chb moves, the coupling portion 21c is switched between the locked state and the unlocked state.

Alternatively, the internal gear 29 may be configured to be directly moved by the operating portion 400. In this case, the internal gear 29 has the function of an interlocking portion.

### Movement of Operating Portion

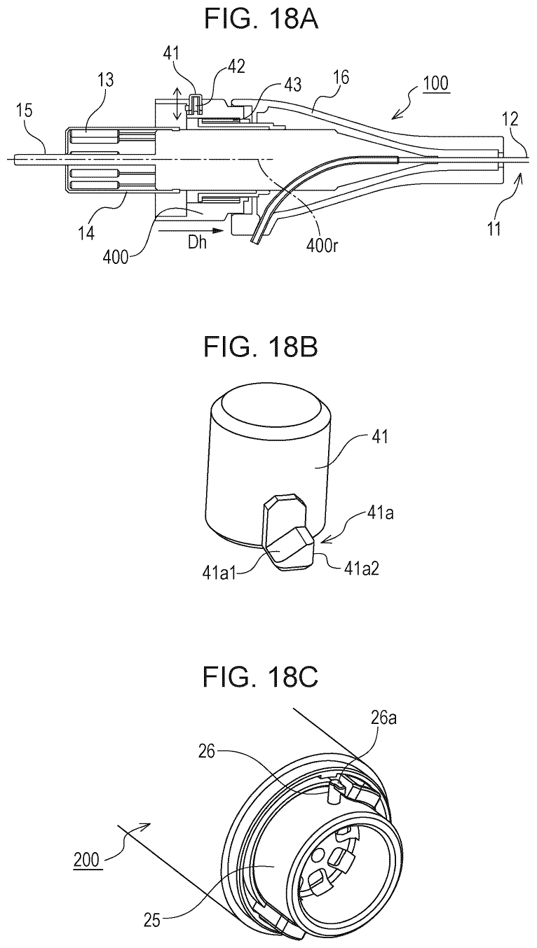

Movement of the operating portion 400 will be described with reference to FIGS. 18A to 18C, 19A to 19C, and 20A to 20C.

In the present embodiment, the operating portion 400 is configured to be movable among the detachment position, the unlock position, and the fixed position in a state where the catheter unit 100 is attached to the base unit 200. The unlock position is located between the detachment position and the fixed position.

In the present embodiment, the operating portion 400 interlocks with the movement of the operating portion 400 between the unlock position and the fixed position, and the state is switched between the first state and the second state.

In the present embodiment, the operating portion 400 is movable between the detachment position and the fixed position by moving in a direction different from the attaching and detaching direction DE. The operating portion 400 moves in a direction that intersects with (preferably, a direction orthogonal to) the attaching and detaching direction DE to move between the detachment position and the fixed position. In the present embodiment, the operating portion 400 rotates around the rotation axis 400r extending in the attaching and detaching direction DE to move between the detachment position and the fixed position. Therefore, operability at the time when a user operates the operating portion 400 is good.

FIGS. 18A to 18C are views that illustrate the catheter unit 100 and the base unit 200. FIG. 18A is a sectional view of the catheter unit 100. FIG. 18B is a perspective view of a button 41. FIG. 18C is a perspective view of the base unit 200.

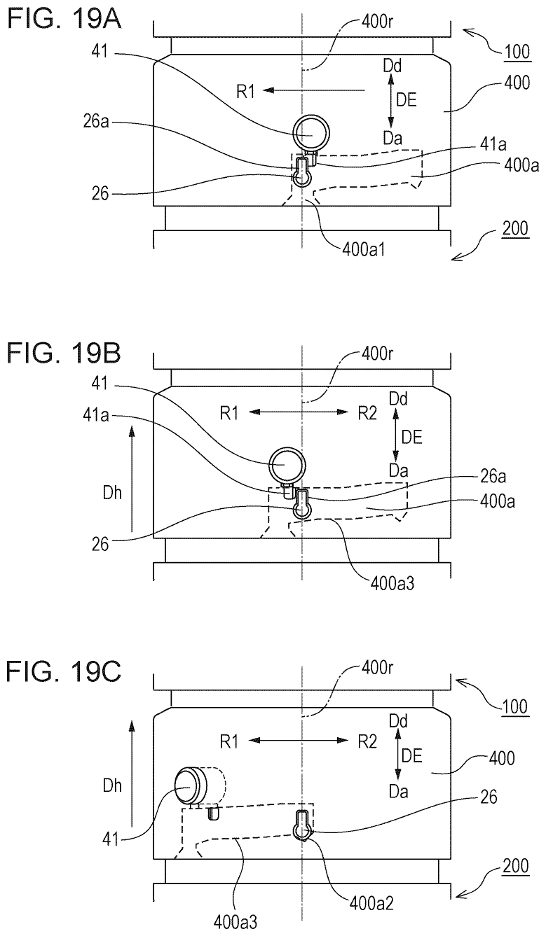

FIGS. 19A to 19C are views that illustrate the operation of the operating portion 400. FIG. 19A is a view that shows a state where the operating portion 400 is at the detachment position. FIG. 19B is a view that shows a state where the operating portion 400 is at the unlock position. FIG. 19C is a view that shows a state where the operating portion 400 is at the fixed position.

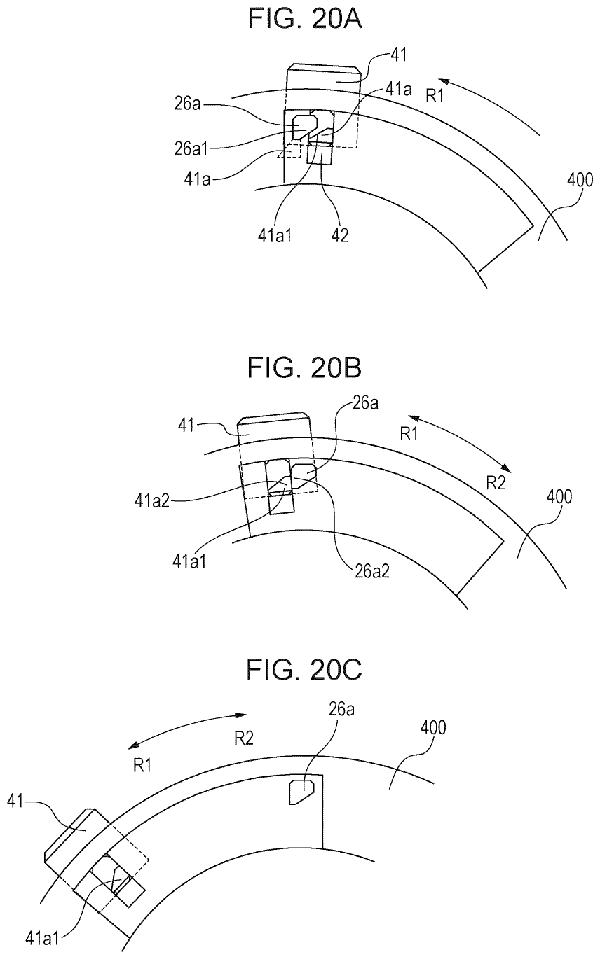

FIGS. 20A to 20C are sectional views that illustrate the operation of the operating portion 400. FIG. 20A is a sectional view that shows a state where the operating portion 400 is at the detachment position. FIG. 20B is a sectional view that shows a state where the operating portion 400 is at the unlock position. FIG. 20C is a sectional view that shows a state where the operating portion 400 is at the fixed position.

When the operating portion 400 is at the fixed position, the coupling portion 21c is in the locked state, and the held portion Wa of the drive wire W is fixed to the corresponding coupling portion 21c (see FIG. 17).

When the operating portion 400 is at the unlock position, the coupling portion 21c is in the unlocked state, and the locking of the held portion Wa of the drive wire W with the coupling portion 21c is unlocked (see FIG. 14). In this state, connection of the drive wire W with the wire drive portion 300 is interrupted. Therefore, at the time when the catheter 11 receives an external force, the bend portion 12 can be freely bent without receiving resistance from the wire drive portion 300.

When the operating portion 400 is at the detachment position, the catheter unit 100 is allowed to be detached from the base unit 200. In a state where the operating portion 400 is at the detachment position, the catheter unit 100 is allowed to be attached to the base unit 200. When the operating portion 400 is at the detachment position, the coupling portion 21c is in the unlocked state, and the locking of the held portion Wa of the drive wire W with the coupling portion 21c is unlocked (see FIG. 14).

As shown in FIG. 18A, the catheter unit 100 includes an operating portion urging spring 43 that urges the operating portion 400, the button 41 serving as a moving member, and a button spring 42 that urges the button 41.

In the present embodiment, the operating portion urging spring 43 is a compression spring. The operating portion 400 is urged in a direction Dh to approach the proximal end cover 16 by the operating portion urging spring 43.

In the present embodiment, the button 41 and the button spring 42 are included in the operating portion 400. When the operating portion 400 moves among the detachment position, the unlock position, and the fixed position, the button 41 and the button spring 42 move together with the operating portion 400.

The button 41 is configured to be movable with respect to the operating portion 400 in a direction that intersects with the direction of the rotation axis 400r of the operating portion 400. The button 41 is urged by the button spring 42 toward outside the catheter unit 100 (a direction to move away from the rotation axis 400r).

As will be described later, movement of the operating portion 400 from the unlock position to the detachment position is restricted by the button 41. When the button 41 is moved with respect to the operating portion 400, the operating portion 400 is allowed to move from the unlock position to the detachment position.

The button 41 has a button protrusion (regulated portion) 41a. The button protrusion 41a has a button slope 41a1 and a restricted surface 41a2.

The base unit 200 includes the base frame 25. The base frame 25 is provided with a lock shaft 26. The lock shaft 26 has a lock protrusion (regulating portion) 26a.

In the present embodiment, a plurality of (two in the present embodiment) the lock shafts 26 is provided. Each of the lock shafts 26 may have the lock protrusion 26a or one or some of the lock shafts 26 may have a lock protrusion 26a.

On the other hand, as shown in FIGS. 12, 18A, 18B, and 18C, a lock groove 400a to be engaged with the lock shaft 26 is provided on the inner side of the operating portion 400. The lock groove 400a extends in a direction different from the attaching and detaching direction DE. In the present embodiment, the lock groove 400a extends in the rotation direction of the operating portion 400. The lock groove 400a may also be regarded as extending in a direction that intersects with (direction orthogonal to) the attaching and detaching direction DE.

When a plurality of the lock shafts 26 is provided, the lock groove 400a is provided for each of the plurality of lock shafts 26.

As shown in FIG. 18A, when the catheter unit 100 is attached to the base unit 200, the lock shaft 26 engages with the lock groove 400a via the entrance 400a1 of the lock groove 400a.

At this time, the operating portion 400 is placed at the detachment position, and the coupling portion 21c is in the unlocked state (see FIG. 14). Therefore, this is a state where fixing of each of the first to ninth drive wires (W11 to W33) with a corresponding one of the first to ninth coupling portions (21c11 to 21c33) is unlocked. As shown in FIG. 19A, the button protrusion 41a is opposite to the lock protrusion 26a.

When the operating portion 400 is rotated in a lock direction R1 in a state where the operating portion 400 is at the detachment position, the slope 41a1 of the button protrusion 41a contacts with a slope 26a1 of the lock protrusion 26a. The button 41 moves toward inside the operating portion 400 (a direction to approach the rotation axis 400r) against the urging force of the button spring 42. Then, the button protrusion 41a climbs over the lock protrusion 26a, and the operating portion 400 moves to the unlock position (see FIG. 19B).

At this time, the coupling portion 21c is in the unlocked state (see FIG. 14). Therefore, this is a state where fixing of each of the first to ninth drive wires (W11 to W33) with a corresponding one of the first to ninth coupling portions (21c11 to 21c33) is unlocked.

In the present embodiment, even when the button 41 is not operated, the operating portion 400 is allowed to be moved from the detachment position to the unlock position. In other words, when the operating portion 400 is moved from the detachment position to the unlock position, a user does not need to operate the button 41.

When the operating portion 400 is rotated in the lock direction R1 in a state where the operating portion 400 is at the unlock position, the operating portion 400 moves to the fixed position. In a state where the operating portion 400 is at the fixed position, a positioning portion 400a2 of the lock groove 400a is placed at a location corresponding to the lock shaft 26. The operating portion 400 is urged in a direction Dh to approach the proximal end cover 16 by the operating portion urging spring 43. As a result, the positioning portion 400a2 is engaged with the lock shaft 26.

In the course in which the operating portion 400 moves from the unlock position to the fixed position, the held portion Wa of the drive wire W is fixed to the coupling portion 21c as described above.

In a state where the operating portion is placed at the fixed position, the coupling portion 21c is in the locked state (see FIG. 17). Therefore, each of the first to ninth drive wires (W11 to W33) is fixed to a corresponding one of the first to ninth coupling portions (21c11 to 21c33). In this state, driving force from the wire drive portion 300 can be transmitted to the bend drive portion 13. In other words, driving force from each of the first to ninth driving sources (M11 to M33) can be transmitted to a corresponding one of the first to ninth drive wires (W11 to W33) via a corresponding one of the first to ninth coupling portions (21c11 to 21c33).

When the operating portion 400 is at the unlock position, a wall 400a3 defining the lock groove 400a is placed on the upstream side of the lock shaft 26 in the detachment direction Dd of the catheter unit 100. When the operating portion 400 is at the fixed position, the positioning portion 400a2 is placed on the upstream side of the lock shaft 26 in the detachment direction Dd. As a result, when the operating portion 400 is at the unlock position or at the fixed position, detaching the catheter unit 100 from the base unit 200 is restricted. On the other hand, when the operating portion 400 is at the detachment position, the entrance 400a1 of the lock groove 400a is placed on the upstream side of the lock shaft 26 in the detachment direction Dd. As a result, detaching the catheter unit 100 from the base unit 200 is allowed.

When the operating portion 400 is rotated in an unlock direction R2 in a state where the operating portion 400 is at the fixed position, the operating portion 400 is placed at the unlock position. In the course in which the operating portion 400 moves from the fixed position to the unlock position, the held portion Wa of the drive wire W is unlocked from the coupling portion 21c as described above.

In a state where the operating portion 400 is placed at the unlock position, the restricted surface 41a2 of the button protrusion 41a contacts with the restriction surface 26a2 of the lock protrusion 26a (see FIG. 19B). In this state, rotating the operating portion 400 in the unlock direction R2 is restricted. Detaching the catheter unit 100 from the base unit 200 is restricted.

When a user pushes the button 41 toward inside the operating portion 400 in a state where the operating portion 400 is placed at the unlock position, the restricted surface 41a2 separates from the restriction surface 26a2, and the button protrusion 41a climbs over the lock protrusion 26a. As a result, the operating portion 400 is allowed to rotate in the unlock direction R2, and the operating portion 400 can be moved from the unlock position to the detachment position.

When the operating portion 400 is placed at the detachment position, the coupling portion 21c is in the unlocked state. Therefore, when the catheter unit 100 is detached from the base unit 200 or attached to the base unit 200, load (for example, resistance received by the coupling portion 21c) applied to the drive wire W is reduced. Therefore, a user is able to easily attach and detach the catheter unit 100.

When the operating portion 400 is placed at the unlock position, detaching the catheter unit 100 from the base unit 200 is restricted, and the coupling portion 21c is in the unlocked state. As described above, when the coupling portion 21c is in the unlocked state, connection of the drive wire W with the wire drive portion 300 is interrupted, and the bend portion 12 can be freely bent without receiving resistance from the wire drive portion 300.

A user is able to stop actuation of the catheter 11 with the wire drive portion 300 by placing the operating portion 400 at the unlock position in a state where the catheter 11 is inserted in a target. In addition, since detaching the catheter unit 100 from the base unit 200 is restricted, a user is able to pull out the catheter 11 from inside the target while holding the base unit 200.

In the configuration of the present embodiment, when the button 41 is not operated, movement of the operating portion 400 from the unlock position to the detachment position is restricted. Therefore, when a user moves the operating portion 400 from the fixed position to the unlock position, erroneous movement of the operating portion 400 to the detachment position is reduced.

In the present embodiment, the number of the lock protrusions 26a and the number of the buttons 41 each are one. However, the medical device 1 may have a plurality of the lock protrusions 26a and a plurality of the buttons 41.

According to the present invention, it is possible to provide a supporting apparatus capable of accurately bending a bend portion and a manufacturing method therefor.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A supporting apparatus configured to support a unit including a bend body, the supporting apparatus comprising:
a drive member configured to rotate around a rotational axis;
a transmission member coupled to the drive member and configured to be moved in a direction of the rotational axis by rotation of the drive member;
a driven member for bending the bend body, the driven member being coupled to the transmission member and configured to be moved in the direction of the rotational axis by movement of the transmission member;
a coupling member;
a first supporting member having a first supporting portion that supports the drive member, a second supporting portion that supports the driven member, and a first engaging portion that is engaged with the coupling member; and
a second supporting member having the same shape as the first supporting member, the second supporting member having (i) a third supporting portion that corresponds to the first supporting portion and that supports the drive member, (ii) a fourth supporting portion that corresponds to the second supporting portion and that supports the driven member, and (iii) a second engaging portion that corresponds to the first engaging portion and that is engaged with the coupling member, the second supporting member being disposed at a location away from the first supporting member in the direction of the rotational axis.

2. The supporting apparatus according to claim 1, wherein the first supporting member has a plurality of the first supporting portions and a plurality of the second supporting portions, and the second supporting member has a plurality of the third supporting portions and a plurality of the fourth supporting portions.

3. The supporting apparatus according to claim 2, wherein the plurality of first supporting portions and the plurality of second supporting portions are disposed so as to surround the first engaging portion, and the plurality of third supporting portions and the plurality of fourth supporting portions are disposed so as to surround the second engaging portion.

4. The supporting apparatus according to claim 1, wherein, when viewed in the direction of the rotational axis, a first straight line connecting a center of the first supporting portion with a center of the second supporting portion intersects with the first engaging portion, and a second straight line connecting a center of the third supporting portion with a center of the fourth supporting portion intersects with the second engaging portion.

5. The supporting apparatus according to claim 4, wherein the second supporting portion is disposed between the first supporting portion and the first engaging portion in a direction of the first straight line, and the fourth supporting portion is disposed between the third supporting portion and the second engaging portion in a direction of the second straight line.

6. The supporting apparatus according to claim 1, wherein the first engaging portion, the first supporting portion, the second supporting portion, the second engaging portion, the third supporting portion, and the fourth supporting portion are holes.

7. The supporting apparatus according to claim 6, wherein a radius of the first engaging portion is greater than a radius of the first supporting portion and is greater than a radius of the second supporting portion, and a radius of the second engaging portion is greater than a radius of the third supporting portion and is greater than a radius of the fourth supporting portion.

8. A manufacturing method for a supporting apparatus configured to support a unit including a bend body, the supporting apparatus including a drive member configured to rotate around a rotational axis, a transmission member coupled to the drive member and configured to be moved in a direction of the rotational axis by rotation of the drive member, a driven member for bending the bend body, the driven member being coupled to the transmission member and configured to be moved in the direction of the rotational axis by movement of the transmission member, and a coupling member, the manufacturing method comprising:
a step of manufacturing a first supporting member having a first supporting portion, a second supporting portion, and a first engaging portion;
a step of manufacturing a second supporting member having (i) a third supporting portion that corresponds to the first supporting portion, (ii) a fourth supporting portion that corresponds to the second supporting portion, and (iii) a second engaging portion that corresponds to the first engaging portion, the second supporting member having the same shape as the first supporting member; and
a step of assembling the drive member, the driven member, the first supporting member, the second supporting member, and the coupling member such that the coupling member is engaged with the first engaging portion and the second engaging portion, the drive member is supported by the first supporting portion and the third supporting portion, and the driven member is supported by the second supporting portion and the fourth supporting portion.

9. The manufacturing method according to claim 8, wherein the first supporting member and the second supporting member are manufactured by injection molding using the same die.

10. The manufacturing method according to claim 8, wherein each set of (i) the first supporting portion and the third supporting portion, (ii) the second supporting portion and the fourth supporting portion, and (iii) the first engaging portion and the second engaging portion is manufactured in a manner such that, in a state where a plurality of materials is stacked, part of the plurality of materials is removed.

11. The manufacturing method according to claim 8, wherein each set of (i) the first supporting portion and the third supporting portion, (ii) the second supporting portion and the fourth supporting portion, and (iii) the first engaging portion and the second engaging portion is manufactured by press working using the same die.
